(19) United States
(12) Patent Application Publication
(10) Pub. No.: US 2025/0032024 A1
(43) Pub. Date: Jan. 30, 2025

(54) MUSCULAR STRENGTH INDEX ESTIMATION DEVICE, MUSCULAR STRENGTH INDEX ESTIMATION SYSTEM, MUSCULAR STRENGTH INDEX ESTIMATION METHOD, AND RECORDING MEDIUM

(71) Applicant: NEC Corporation, Minato-ku, Tokyo (JP)

(72) Inventors: Chenhui HUANG, Tokyo (JP); Fumiyuki NIHEY, Tokyo (JP); Hiroshi KAJITANI, Tokyo (JP); Yoshitaka NOZAKI, Tokyo (JP); Kenichiro FUKUSHI, Tokyo (JP); Kenichiro FUKUSHI, Tokyo (JP)

(73) Assignee: NEC Corporation, Minato-ku, Tokyo (JP)

(21) Appl. No.: 18/716,281

(22) PCT Filed: Dec. 27, 2021
(86) PCT No.: PCT/JP2021/048549

§ 371 Date: Jun. 4, 2024

(51) Int. Cl. A61B 5/22; A61B 5/00; A61B 5/11; G16H 10/20

(52) U.S. Cl. CPC A61B 5/225; A61B 5/112; A61B 5/486; A61B 5/7267; A61B 5/742; G16H 10/20

(57) ABSTRACT

Provided is a muscular strength index estimation device that includes a data acquisition unit that acquires feature amount data including a feature amount, the feature amount being extracted from a feature of gait of a user and being used for estimation of a muscular strength index of the user, a storage unit that stores an estimation model that outputs the muscular strength index in accordance with an input of the feature amount data, an estimation unit that inputs the acquired feature amount data to the estimation model to estimate the muscular strength index of the user, and an output unit that outputs information regarding the estimated muscular strength index.

### TECHNICAL FIELD

The present disclosure relates to a muscular strength index estimation device or the like that estimates a muscular strength index using data regarding gait.

### BACKGROUND ART

With increasing interest in healthcare, services for providing information according to features (also referred to as gait) included in a gait pattern have attracted attention. For example, a technique for analyzing gait based on sensor data measured by a sensor mounted on footwear such as shoes has been developed. In time-series data of the sensor data, a feature of a gait event (also referred to as a gait event) related to a physical condition appears.

PTL 1 discloses a device that detects an abnormality of a foot based on features of gait of a walker. The device of PTL 1 extracts a characteristic gait feature amount in gait of a walker wearing footwear by using data acquired from a sensor installed in the footwear. The device of PTL 1 detects an abnormality of the walker walking wearing footwear based on the extracted gait feature amount. For example, the device of PTL 1 extracts a characteristic part regarding hallux valgus from gait waveform data for one gait cycle. The device of PTL 1 estimates the progression state of hallux valgus using the extracted gait feature amount of the characteristic part.

For example, the grip strength is an index for evaluating the total muscular strength of the whole body (also referred to as total whole body muscular strength), and can also be an important index for evaluating the frailty or the risk of falling. NPL 1 discloses that there is a high correlation between the grip strength and the knee extension strength. The knee extension strength is an index of the muscular strength of a muscle group for extending the knee, such as quadriceps femoris.

PTL 2 discloses a leg muscular strength estimation device that estimates information related to leg muscular strength of a subject using measurement data obtained by a communication type grip strength meter. The device of PTL 2 calculates the maximum leg extension muscular strength/weight data of an individual using a grip strength value data measured with the communication type grip strength meter and personal data input from a personal data input means. The device of PTL 2 calculates falling age data that is the age at which the possibility of falling increases using the maximum leg extension muscular strength/weight data of the individual and the personal data.

### CITATION LIST

### Patent Literature

PTL 1: WO 2021/140658 A1
PTL 2: JP 2014-221139 A

### Non Patent Literature

NPL 1: R. Bohannon, et al., “Grip and Knee Extension Muscle Strength Reflect a Common Construct among Adults”, Muscle Nerve, 2012 October, vol. 46 (4), pp. 555-558.

### SUMMARY OF INVENTION

### Technical Problem

In the method of PTL 1, the progression state of hallux valgus is estimated using the gait feature amount of the characteristic part extracted from the data acquired from the sensor installed in the footwear. PTL 1 does not disclose that the grip strength is estimated using the gait feature amount of the characteristic part extracted from the data acquired from the sensor installed in the footwear.

In the method of PTL 2, the maximum leg extension muscular strength/weight data of the individual is calculated using the measurement data by the communication type grip strength meter and the input personal data. In the method of PTL 2, it is necessary to measure the grip strength using the communication type grip strength meter, and thus, it is not possible to appropriately estimate the muscular strength index in everyday life.

An object of the present disclosure is to provide a muscular strength index estimation device and the like capable of appropriately estimating a muscular strength index in everyday life.

### Solution to Problem

A muscular strength index estimation device according to one aspect of the present disclosure includes, a data acquisition unit that acquires feature amount data including a feature amount, the feature amount being extracted from a feature of gait of a user and being used for estimation of a muscular strength index of the user, a storage unit that stores an estimation model that outputs the muscular strength index in accordance with an input of the feature amount data, an estimation unit that inputs the acquired feature amount data to the estimation model to estimate the muscular strength index of the user, and an output unit that outputs information regarding the estimated muscular strength index.

A muscular strength index estimation method according to one aspect of the present disclosure includes acquiring feature amount data including a feature amount, the feature amount being extracted from a feature of gait of a user and being used for estimation of a muscular strength index of the user, inputting the acquired feature amount data to an estimation model that outputs the muscular strength index in accordance with an input of the feature amount data to estimate the muscular strength index of the user, and outputting information regarding the estimated muscular strength index.

A program according to one aspect of the present disclosure causes a computer to execute, processing of acquiring feature amount data including a feature amount, the feature amount being extracted from a feature of gait of a user and being used for estimation of a muscular strength index of the user, processing of inputting the acquired feature amount data to an estimation model that outputs the muscular strength index in accordance with an input of the feature amount data to estimate the muscular strength index of the user, and processing of outputting information regarding the estimated muscular strength index.

### Advantageous Effects of Invention

According to the present disclosure, it is possible to provide a muscular strength index estimation device and the like capable of appropriately estimating a muscular strength index in everyday life.

### BRIEF DESCRIPTION OF DRAWINGS

FIG. 1 is a block diagram illustrating an example of a configuration of a muscular strength index estimation system according to a first example embodiment.

FIG. 2 is a block diagram illustrating an example of a configuration of a gait measurement device included in the muscular strength index estimation system according to the first example embodiment. 10

FIG. 3 is a conceptual diagram illustrating an arrangement example of the gait measurement device according to the first example embodiment.

FIG. 4 is a conceptual diagram for describing an example of a relationship between a local coordinate system and a world coordinate system set in the gait measurement device according to the first example embodiment.

FIG. 5 is a conceptual diagram for describing human body planes used in the description regarding the gait measurement device according to the first example embodiment.

FIG. 6 is a conceptual diagram for describing a gait cycle used in the description regarding the gait measurement device according to the first example embodiment.

FIG. 7 is a graph for describing an example of time-series data of sensor data measured by the gait measurement device according to the first example embodiment.

FIG. 8 is a diagram for describing an example of normalization of gait waveform data extracted from the time-series data of the sensor data measured by the gait measurement device according to the first example embodiment.

FIG. 9 is a conceptual diagram for describing an example of a gait phase cluster from which a feature amount data generation unit of the gait measurement device according to the first example embodiment extracts a feature amount.

FIG. 10 is a block diagram illustrating an example of a configuration of a muscular strength index estimation device included in the muscular strength index estimation system according to the first example embodiment.

FIG. 11 is a table related to specific examples of the feature amount extracted by the gait measurement device included in the muscular strength index estimation system according to the first example embodiment for estimating the grip strength of a male.

FIG. 12 is a graph illustrating a correlation between a feature amount M1 extracted by the gait measurement device included in the muscular strength index estimation system according to the first example embodiment and a measured grip strength of a male.

FIG. 13 is a graph illustrating a correlation between a feature amount M2 extracted by the gait measurement device included in the muscular strength index estimation system according to the first example embodiment and a measured grip strength of a male.

FIG. 14 is a graph illustrating a correlation between a feature amount M3 extracted by the gait measurement device included in the muscular strength index estimation system according to the first example embodiment and a measured grip strength of a male.

FIG. 15 is a graph illustrating a correlation between a feature amount M4 extracted by the gait measurement device included in the muscular strength index estimation system according to the first example embodiment and a measured grip strength of a male.

FIG. 16 is a block diagram illustrating an estimation example of a grip strength (muscular strength index) of a male by the muscular strength index estimation device included in the muscular strength index estimation system according to the first example embodiment.

FIG. 17 is a graph illustrating a correlation between an estimation value of grip strength estimated using an estimation model generated by machine learning with age and height as explanatory variables and a measurement value of grip strength.

FIG. 18 is a graph illustrating a correlation between an estimation value of grip strength estimated by the muscular strength index estimation device included in the muscular strength index estimation system according to the first example embodiment and a measurement value of grip strength.

FIG. 19 is a table related to specific examples of the feature amount extracted by the gait measurement device included in the muscular strength index estimation system according to the first example embodiment for estimating the grip strength of a female.

FIG. 20 is a graph illustrating a correlation between a feature amount F1 extracted by the gait measurement device included in the muscular strength index estimation system according to the first example embodiment and a measured grip strength of a female.

FIG. 21 is a graph illustrating a correlation between a feature amount F2 extracted by the gait measurement device included in the muscular strength index estimation system according to the first example embodiment and a measured grip strength of a female.

FIG. 22 is a graph illustrating a correlation between a feature amount F3 extracted by the gait measurement device included in the muscular strength index estimation system according to the first example embodiment and a measured grip strength of a female.

FIG. 23 is a block diagram illustrating an estimation example of a grip strength (muscular strength index) of a female by the muscular strength index estimation device included in the muscular strength index estimation system according to the first example embodiment.

FIG. 24 is a graph illustrating a correlation between an estimation value of grip strength estimated using an estimation model generated by machine learning with age and height as explanatory variables and a measurement value of grip strength.

FIG. 25 is a graph illustrating a correlation between an estimation value of grip strength estimated by the muscular strength index estimation device included in the muscular strength index estimation system according to the first example embodiment and a measurement value of grip strength.

FIG. 26 is a block diagram illustrating an estimation example of a knee extension strength (muscular strength index) by the muscular strength index estimation device included in the muscular strength index estimation system according to the first example embodiment.

FIG. 27 is a flowchart for describing an example of an operation of the gait measurement device included in the muscular strength index estimation system according to the first example embodiment.

FIG. 28 is a flowchart for describing an example of an operation of the muscular strength index estimation device included in the muscular strength index estimation system according to the first example embodiment.

FIG. 29 is a conceptual diagram for describing an application example of the muscular strength index estimation system according to the first example embodiment.

FIG. 30 is a conceptual diagram for describing an application example of the muscular strength index estimation system according to the first example embodiment.

FIG. 31 is a block diagram illustrating an example of a configuration of a machine learning system according to a second example embodiment.

FIG. 32 is a block diagram illustrating an example of a configuration of a machine learning device included in the machine learning system according to the second example embodiment.

FIG. 33 is a conceptual diagram for describing an example of machine learning by the machine learning device included in the machine learning system according to the second example embodiment.

FIG. 34 is a conceptual diagram for describing another example of machine learning by the machine learning device included in the machine learning system according to the second example embodiment.

FIG. 35 is a block diagram illustrating an example of a configuration of a muscular strength index estimation device according to a third example embodiment.

FIG. 36 is a block diagram illustrating an example of a hardware configuration that executes control and processing of each example embodiment.

### EXAMPLE EMBODIMENT

Hereinafter, example embodiments of the present invention will be described with reference to the drawings. However, the example embodiments described below have technically preferable limitations for carrying out the present invention, but do not limit the scope of the invention to those described below. In all the drawings used in the description of the example embodiments below, the same reference numerals are given to the same parts unless there is a particular reason. In the example embodiments described below, repeated description of similar configurations and operations may be omitted.

### First Example Embodiment

First, a muscular strength index estimation system according to a first example embodiment will be described with reference to the drawings. The muscular strength index estimation system according to the present example embodiment measures sensor data regarding a motion of a foot according to gait of a user. The muscular strength index estimation system of the present example embodiment estimates the muscular strength index of the user using the measured sensor data. In the present example embodiment, an example of estimating the grip strength and the knee extension strength is described as the muscular strength index. The sensor data is not limited to the sensor data regarding the motion of the foot, and it is sufficient if the sensor data includes a feature regarding the gait. For example, the sensor data may be sensor data including a feature regarding the gait measured using motion capture, smart apparel, or the like.

### Configuration

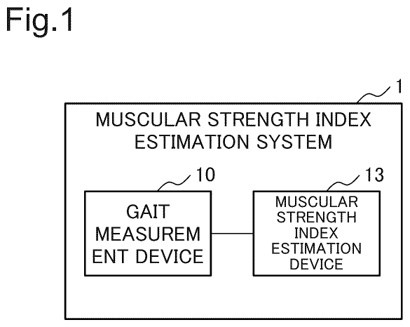

FIG. 1 is a block diagram illustrating an example of a configuration of a muscular strength index estimation system 1 according to the present example embodiment. The muscular strength index estimation system 1 includes a gait measurement device 10 and a muscular strength index estimation device 13. In the present example embodiment, an example in which the gait measurement device 10 and the muscular strength index estimation device 13 are configured as separate hardware will be described. For example, the gait measurement device 10 is installed in footwear or the like of a subject (user) who is a target for estimation of the muscular strength index. For example, the function of the muscular strength index estimation device 13 is installed in a mobile terminal carried by a subject (user). Hereinafter, configurations of the gait measurement device 10 and the muscular strength index estimation device 13 will be individually described.

### [Gait Measurement Device]

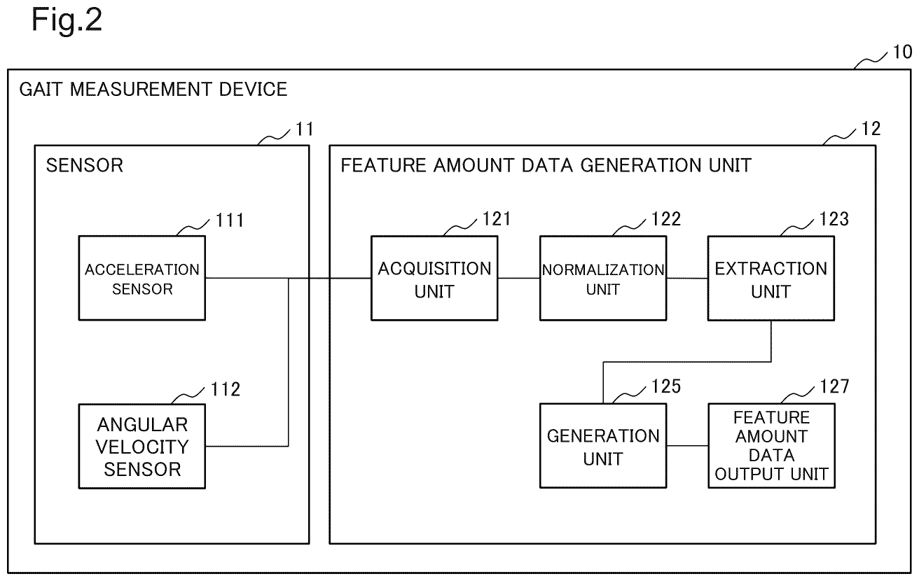

FIG. 2 is a block diagram illustrating an example of a configuration of the gait measurement device 10. The gait measurement device 10 includes a sensor 11 and a feature amount data generation unit 12. In the present example embodiment, an example in which the sensor 11 and the feature amount data generation unit 12 are integrated will be described. The sensor 11 and the feature amount data generation unit 12 may be provided as separate devices.

As illustrated in FIG. 2, the sensor 11 includes an acceleration sensor 111 and an angular velocity sensor 112. FIG. 2 illustrates an example in which the acceleration sensor 111 and the angular velocity sensor 112 are included in the sensor 11. The sensor 11 may include a sensor other than the acceleration sensor 111 and the angular velocity sensor 112. Sensors other than the acceleration sensor 111 and the angular velocity sensor 112 that can be included in the sensor 11 will not be described.

The acceleration sensor 111 is a sensor that measures accelerations (also referred to as spatial accelerations) in three axial directions. The acceleration sensor 111 measures acceleration (also referred to as spatial acceleration) as a physical quantity related to the motion of the foot. The acceleration sensor 111 outputs the measured acceleration to the feature amount data generation unit 12. For example, a sensor of a piezoelectric type, a piezoresistive type, a capacitance type, or the like can be used as the acceleration sensor 111. The measurement method of the sensor used as the acceleration sensor 111 is not limited as long as the sensor can measure the acceleration.

The angular velocity sensor 112 is a sensor that measures angular velocities (also referred to as spatial angular velocities) around three axes. The angular velocity sensor 112 measures angular velocity (also referred to as spatial angular velocity) as a physical quantity related to the motion of the foot. The angular velocity sensor 112 outputs the measured angular velocity to the feature amount data generation unit 12. For example, a sensor of a vibration type, a capacitance type, or the like can be used as the angular velocity sensor 112. The measurement method of the sensor used as the angular velocity sensor 112 is not limited as long as the sensor can measure the angular velocity.

The sensor 11 is achieved by, for example, an inertial measurement device that measures the acceleration and the angular velocity. The inertial measurement unit (IMU) is an example of the inertial measurement device. The IMU includes the acceleration sensor 111 that measures the accelerations in the three axial directions and the angular velocity sensor 112 that measures the angular velocities around the three axes. The sensor 11 may be achieved by an inertial measurement device such as a vertical gyro (VG) or an attitude heading (AHRS). The sensor 11 may be achieved by a global positioning system/inertial navigation system (GPS/INS). The sensor 11 may be achieved by a device other than the inertial measurement device as long as it can measure a physical quantity related to the motion of the foot.

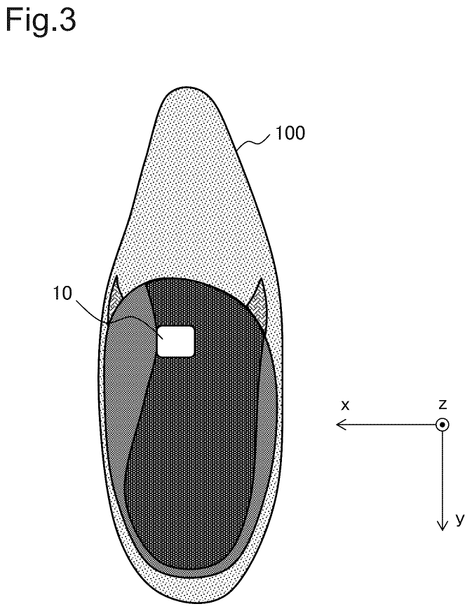

FIG. 3 is a conceptual diagram illustrating an example in which the gait measurement device 10 is arranged in a shoe 100 of a right foot. In the example of FIG. 3, the gait measurement device 10 is installed at a position corresponding to the back side of the arch of the foot. For example, the gait measurement device 10 is arranged on an insole inserted into the shoe 100. For example, the gait measurement device 10 may be arranged on the bottom surface of the shoe 100. For example, the gait measurement device 10 may be embedded in the main body of the shoe 100. The gait measurement device 10 may be detachable from the shoe 100 or may not be detachable from the shoe 100. The gait measurement device 10 may be installed at a position other than the back side of the arch of the foot as long as sensor data regarding the motion of the foot can be measured. The gait measurement device 10 may be installed on a sock worn by the user or a decorative article such as an anklet worn by the user. The gait measurement device 10 may be directly attached to the foot or may be embedded in the foot. FIG. 3 illustrates an example in which the gait measurement device 10 is installed in the shoe 100 of the right foot. The gait measurement device 10 may be installed in the shoes 100 of both feet.

In the example of FIG. 3, a local coordinate system including an x axis in a left-right direction, a y axis in a front-rear direction, and a z axis in an up-down direction is set with reference to the gait measurement device 10 (sensor 11). In the x-axis, the left side is positive, in the y-axis, the rear side is positive, and in the z-axis, the upper side is positive. The direction of the axis set in the sensor 11 may be the same for the left and right feet, or may be different for the left and right feet. For example, in a case where the sensors 11 produced with the same specs are arranged in the left and right shoes 100, the up-down directions (directions in the Z-axis direction) of the sensors 11 arranged in the left and right shoes 100 are the same. In this case, the three axes of the local coordinate system set in the sensor data derived from the left foot and the three axes of the local coordinate system set in the sensor data derived from the right foot are the same for the left and right.

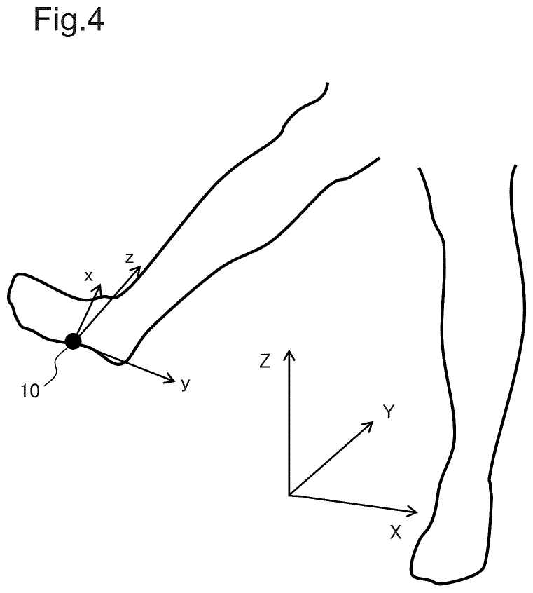

FIG. 4 is a conceptual diagram for describing a local coordinate system (x-axis, y-axis, z-axis) set in the gait measurement device 10 (sensor 11) installed on the back side of the arch of the foot and a world coordinate system (X-axis, Y-axis, Z-axis) set with respect to the ground. In the world coordinate system (X-axis, Y-axis, Z-axis), in a state where the user facing the traveling direction is upright, the lateral direction of the user is set to the X-axis direction (the leftward direction is positive), the direction of the back surface of the user is set to the Y-axis direction (the rearward direction is positive), and the gravity direction is set to the Z-axis direction (the vertically upward direction is positive). The example of FIG. 4 conceptually illustrates the relationship between the local coordinate system (x-axis, y-axis, z-axis) and the world coordinate system (X-axis, Y-axis, Z-axis), and does not accurately illustrate the relationship between the local coordinate system and the world coordinate system that varies depending on the gait of the user.

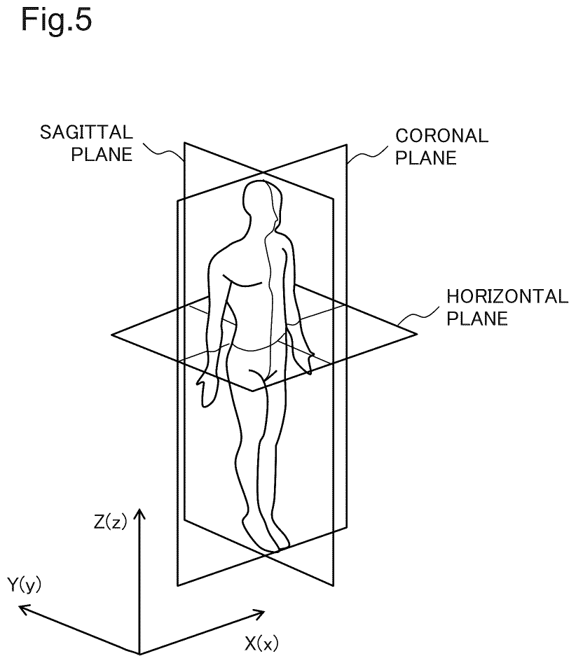

FIG. 5 is a conceptual diagram for describing planes (also referred to as human body planes) set for a human body. In the present example embodiment, a sagittal plane dividing the body into left and right, a coronal plane dividing the body into front and rear, and a horizontal plane dividing the body horizontally are defined. As illustrated in FIG. 5, the world coordinate system and the local coordinate system coincide with each other in the upright state with the center line of the foot oriented in the traveling direction. In the present example embodiment, rotation in the sagittal plane with the x-axis as the rotation axis is defined as roll, rotation in the coronal plane with the y-axis as the rotation axis is defined as pitch, and rotation in the horizontal plane with the z-axis as the rotation axis is defined as yaw. A rotation angle in the sagittal plane with the x-axis as the rotation axis is defined as a roll angle, a rotation angle in the coronal plane with the y-axis as the rotation axis is defined as a pitch angle, and a rotation angle in the horizontal plane with the z-axis as the rotation axis is defined as a yaw angle.

As illustrated in FIG. 2, the feature amount data generation unit 12 (also referred to as a feature amount data generation device) includes an acquisition unit 121, a normalization unit 122, an extraction unit 123, a generation unit 125, and a feature amount data output unit 127. For example, the feature amount data generation unit 12 is achieved by a microcomputer or a microcontroller that performs overall control and data processing of the gait measurement device 10. For example, the feature amount data generation unit 12 includes a central processing unit (CPU), random access memory (RAM), read only memory (ROM), flash memory, and the like. The feature amount data generation unit 12 controls the acceleration sensor 111 and the angular velocity sensor 112 to measure the angular velocity and the acceleration. For example, the feature amount data generation unit 12 may be mounted on a mobile terminal (not illustrated) carried by a subject (user).

The acquisition unit 121 acquires the accelerations in the three axial directions from the acceleration sensor 111. The acquisition unit 121 acquires the angular velocities around the three axes from the angular velocity sensor 112. For example, the acquisition unit 121 performs analog-to-digital conversion (AD conversion) on the acquired physical quantities (analog data) such as the angular velocity and the acceleration. The physical quantities (analog data) measured by the acceleration sensor 111 and the angular velocity sensor 112 may be converted into digital data in each of the acceleration sensor 111 and the angular velocity sensor 112. The acquisition unit 121 outputs the converted digital data (also referred to as sensor data) to the normalization unit 122. The acquisition unit 121 may be configured to cause a storage unit, which is not illustrated, to store the sensor data. The sensor data includes at least acceleration data converted into digital data and angular velocity data converted into digital data. The acceleration data includes acceleration vectors in the three axial directions. The angular velocity data includes angular velocity vectors around the three axes. The acceleration data and the angular velocity data are associated with acquisition times of the data. The acquisition unit 121 may add correction such as a mounting error, temperature correction, and linearity correction to the acceleration data and the angular velocity data.

The normalization unit 122 acquires the sensor data from the acquisition unit 121. The normalization unit 122 extracts time-series data (also referred to as gait waveform data) for one gait cycle from the time-series data of the accelerations in the three axial directions and the angular velocities around the three axes included in the sensor data. The normalization unit 122 normalizes (also referred to as first-normalizes) the time of the extracted gait waveform data for one gait cycle to a gait cycle of 0 to 100% (percent). Timing such as 1% or 10% included in the gait cycle of 0 to 100% is also referred to as a gait phase. The normalization unit 122 normalizes (also referred to as second-normalizes) the first-normalized gait waveform data for one gait cycle in such a way that the stance phase becomes 60% and the swing phase becomes 40%. The stance phase is a period in which at least a part of the back side of the foot is in contact with the ground. The swing phase is a period in which the back side of the foot is away from the ground. When the gait waveform data is subjected to the second normalization, it is possible to suppress a deviation of the gait phase from which the feature amount is extracted from fluctuating due to the effect of disturbance.

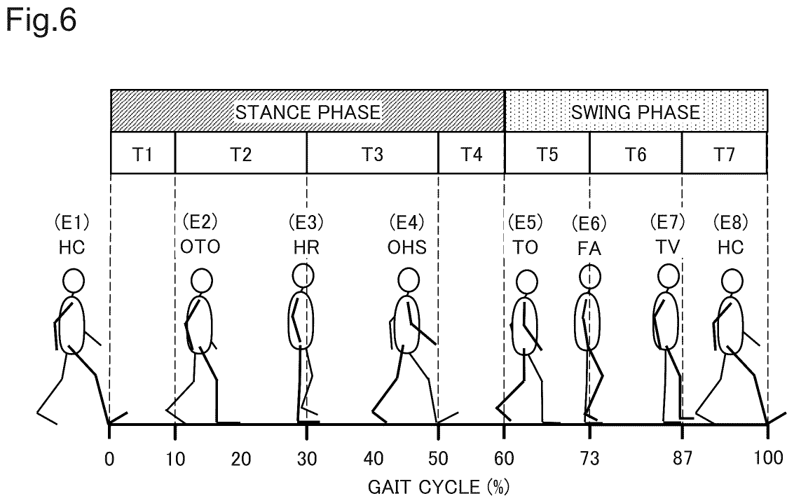

FIG. 6 is a conceptual diagram for describing one gait cycle based on the right foot. One gait cycle based on the left foot is similar to that of the right foot. The horizontal axis in FIG. 6 is one gait cycle of the right foot with a time point at which the heel of the right foot lands on the ground as a starting point and a time point at which the heel of the right foot next lands on the ground as an ending point. The horizontal axis in FIG. 6 is first-normalized with one gait cycle as 100%. The horizontal axis in FIG. 6 is second-normalized such that the stance phase is 60% and the swing phase is 40%. The one gait cycle of one foot is roughly divided into the stance phase in which at least a part of the back side of the foot is in contact with the ground and the swing phase in which the back side of the foot is away from the ground. The stance phase is further subdivided into a load response period T1, a mid-stance period T2, a terminal stance period T3, and a pre-swing period T4. The swing phase is further subdivided into an initial swing period T5, a mid-swing period T6, and a terminal swing period T7. FIG. 6 is an example, and does not limit the periods constituting the one gait cycle, the names of these periods, and the like.

As illustrated in FIG. 6, in gait, a plurality of events (also referred to as gait events) occur. E1 indicates an event in which the heel of the right foot touches the ground (heel contact (HC)). E2 indicates an event in which the toe of the left foot is away from the ground with the sole of the right foot in contact with the ground (opposite toe off (OTO)). E3 indicates an event in which the heel of the right foot rises with the sole of the right foot in contact with the ground (heel rise (HR)). E4 is an event in which the heel of the left foot touches the ground (opposite heal strike (OHS)). E5 indicates an event in which the toe of the right foot is away from the ground with the sole of the left foot in contact with the ground (toe off (TO)). E6 indicates an event in which the left foot and the right foot cross with the sole of the left foot in contact with the ground (foot adjacent (FA). E7 indicates an event in which the tibia of the right foot is approximately perpendicular to the ground with the sole of the left foot in contact with the ground (tibia vertical (TV)). E8 indicates an event in which the heel of the right foot touches the ground (heel contact (HC)). E8 corresponds to the ending point of the gait cycle starting from E1 and corresponds to the starting point of the next gait cycle. FIG. 6 is an example, and does not limit the events that occur during gait or the names of these events.

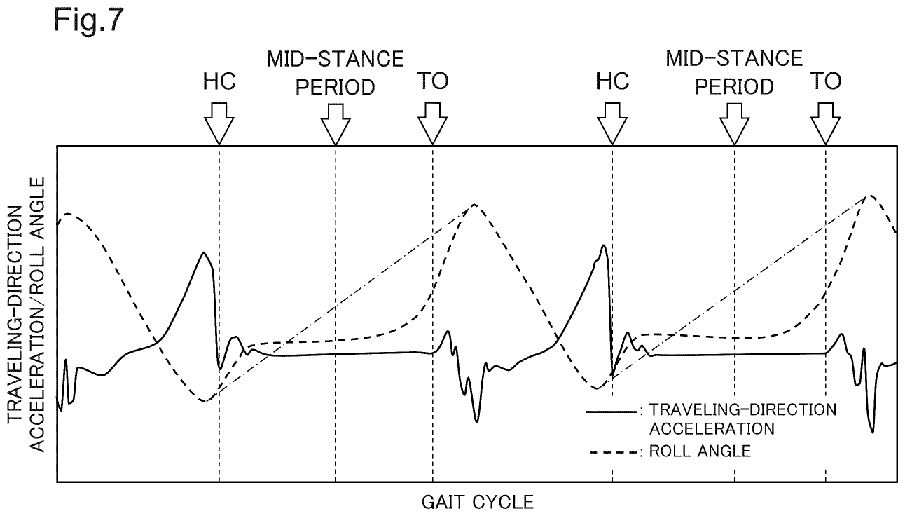

FIG. 7 is a diagram for describing an example of detecting the heel contact HC and the toe off TO from the time-series data (solid line) of the traveling-direction acceleration (Y-direction acceleration). The timing of the heel contact HC is the timing of the minimum peak immediately after the maximum peak appearing in the time-series data of the traveling-direction acceleration (Y-direction acceleration). The maximum peak serving as a mark of the timing of the heel contact HC corresponds to the largest peak of the gait waveform data for one gait cycle. A section between the consecutive pieces of heel contact HC is one gait cycle. The timing of the toe off TO is the timing of rising of the maximum peak appearing after the period of the stance phase in which the fluctuation does not appear in the time-series data of the traveling-direction acceleration (Y-direction acceleration). FIG. 6 also illustrates time-series data (broken line) of the roll angle (angular velocity around the X axis). The timing at the midpoint between the timing at which the roll angle is minimum and the timing at which the roll angle is maximum corresponds to the mid-stance period. For example, parameters (also referred to as gait parameters) such as gait speed, stride, circumduction, medial rotation/external rotation, and plantarflexion/dorsiflexion can be obtained with reference to the mid-stance period.

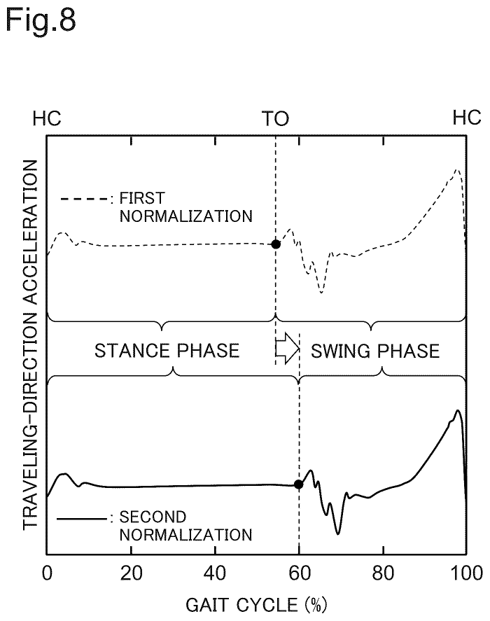

FIG. 8 is a diagram for describing an example of the gait waveform data normalized by the normalization unit 122. The normalization unit 122 detects the heel contact HC and the toe off TO from the time-series data of the traveling-direction acceleration (Y-direction acceleration). The normalization unit 122 extracts the section between the consecutive pieces of heel contact HC as gait waveform data for one gait cycle. The normalization unit 122 converts the horizontal axis (time axis) of the gait waveform data for one gait cycle into a gait cycle of 0 to 100% by the first normalization. In FIG. 7, the gait waveform data after the first normalization is indicated by the broken line. In the gait waveform data (broken line) after the first normalization, the timing of the toe off TO deviates from 60%.

In the example of FIG. 8, the normalization unit 122 normalizes a section from the heel contact HC in which the gait phase is 0% to the toe off TO subsequent to the heel contact HC to 0 to 60%. The normalization unit 122 normalizes a section from the toe off TO to the heel contact HC in which the gait phase is 100% subsequent to the toe off TO to 60 to 100%. As a result, the gait waveform data for one gait cycle is normalized to a section (stance phase) in which the gait cycle is 0 to 60% and a section (swing phase) in which the gait cycle is 60 to 100%. In FIG. 8, the gait waveform data after the second normalization is indicated by the solid line. In the gait waveform data (solid line) after the second normalization, the timing of the toe off TO coincides with 60%.

FIGS. 7 to 8 illustrate examples in which the gait waveform data for one gait cycle is extracted/normalized based on the traveling-direction acceleration (Y-direction acceleration). With respect to acceleration other than the traveling-direction acceleration (Y-direction acceleration)/angular velocity, the normalization unit 122 extracts/normalizes the gait waveform data for one gait cycle in accordance with the gait cycle of the traveling-direction acceleration (Y-direction acceleration). The normalization unit 122 may generate time-series data of the angles around the three axes by integrating the time-series data of the angular velocities around the three axes. In that case, also regarding the angles around the three axes, the normalization unit 122 extracts/normalizes the gait waveform data for one gait cycle in accordance with the gait cycle of the traveling-direction acceleration (Y-direction acceleration).

The normalization unit 122 may extract/normalize the gait waveform data for one gait cycle based on acceleration other than the traveling-direction acceleration (Y-direction acceleration)/angular velocity (the drawings are omitted). For example, the normalization unit 122 may detect the heel contact HC and the toe off TO from the time-series data of the perpendicular-direction acceleration (Z-direction acceleration). The timing of the heel contact HC is the timing of the steep minimum peak appearing in the time-series data of the perpendicular-direction acceleration (Z-direction acceleration). At the timing of the steep minimum peak, the value of the perpendicular-direction acceleration (Z-direction acceleration) becomes substantially zero. The minimum peak serving as a mark of the timing of the heel contact HC corresponds to the smallest peak of the gait waveform data for one gait cycle. A section between the consecutive pieces of heel contact HC is one gait cycle. The timing of the toe off TO is a timing of an inflection point in the middle of a gradual increase of the time-series data of the perpendicular-direction acceleration (Z-direction acceleration) after passing through a section with a small fluctuation after the maximum peak immediately after the heel contact HC. The normalization unit 122 may extract/normalize the gait waveform data for one gait cycle based on both the traveling-direction acceleration (Y-direction acceleration) and the perpendicular-direction acceleration (Z-direction acceleration). The normalization unit 122 may extract/normalize the gait waveform data for one gait cycle based on the acceleration other than the traveling-direction acceleration (Y-direction acceleration) and the perpendicular-direction acceleration (Z-direction acceleration), the angular velocity, the angle, and the like.

The extraction unit 123 acquires the gait waveform data for one gait cycle normalized by the normalization unit 122. The extraction unit 123 extracts a feature amount used for estimation of the muscular strength index from the gait waveform data for one gait cycle. The extraction unit 123 extracts a feature amount for each gait phase cluster from a gait phase cluster obtained by integrating temporally continuous gait phases based on a preset condition. The gait phase cluster includes at least one gait phase. The gait phase cluster also includes a single gait phase. The gait waveform data and the gait phase from which the feature amount used for estimating the muscular strength index is extracted will be described below.

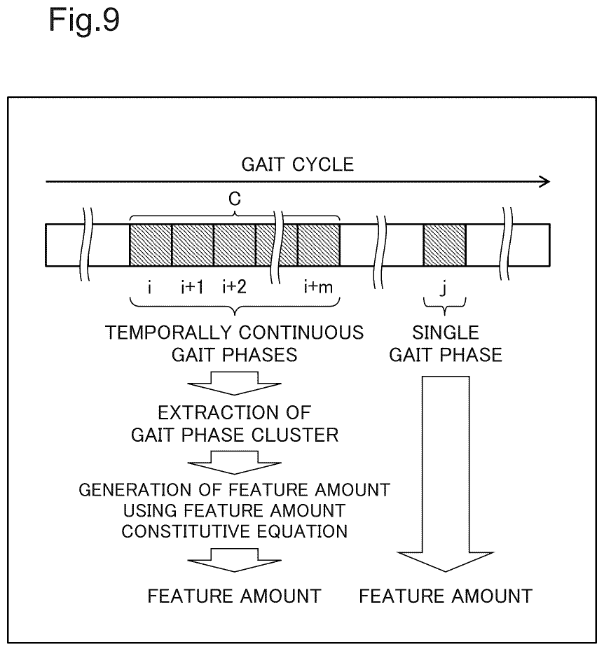

FIG. 9 is a conceptual diagram for describing extraction of a feature amount for estimating a muscular strength index from gait waveform data for one gait cycle. For example, the extraction unit 123 extracts temporally continuous gait phases i to i+m as a gait phase cluster C (i and m are natural numbers). The gait phase cluster C includes m gait phases (components). That is, the number of gait phases (components) (also referred to as the number of components) constituting the gait phase cluster C is m. FIG. 9 illustrates an example in which the gait phase has an integer value, but the gait phase may be subdivided into decimal places. When the gait phase is subdivided into decimal places, the number of components of the gait phase cluster C is a number related to the number of pieces of data in the section of the gait phase cluster. The extraction unit 123 extracts a feature amount from each of the gait phases i to i+m. In a case where the gait phase cluster C includes a single gait phase j, the extraction unit 123 extracts a feature amount from the single gait phase j (j is a natural number).

The generation unit 125 applies a feature amount constitutive equation to the feature amount (first feature amount) extracted from each of the gait phases constituting the gait phase cluster to generate the feature amount (second feature amount) of the gait phase cluster. The feature amount constitutive equation is a preset calculation expression for generating the feature amount of the gait phase cluster. For example, the feature amount constitutive equation is a calculation expression related to four arithmetic operations. For example, the second feature amount calculated using the feature amount constitutive equation is an integral average value, an arithmetic average value, a slope, a variation, or the like of the first feature amount in each gait phase included in the gait phase cluster. For example, the generation unit 125 applies the calculation expression for calculating the slope and variation of the first feature amount extracted from each of the gait phases constituting the gait phase cluster as the feature amount constitutive equation. For example, in a case where the gait phase cluster includes a single gait phase, it is not possible to calculate the slope or variation, and thus, it is sufficient to use the feature amount constitutive equation for calculating an integral average value, an arithmetic average value, or the like.

The feature amount data output unit 127 outputs the feature amount data for each gait phase cluster generated by the generation unit 125. The feature amount data output unit 127 outputs the generated feature amount data of the gait phase cluster to the muscular strength index estimation device 13 using the feature amount data.

### [Muscular Strength Index Estimation Device]

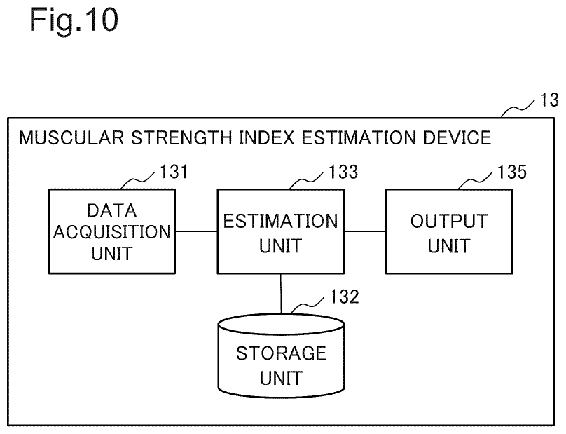

FIG. 10 is a block diagram illustrating an example of a configuration of the muscular strength index estimation device 13. The muscular strength index estimation device 13 includes a data acquisition unit 131, a storage unit 132, an estimation unit 133, and an output unit 135.

The data acquisition unit 131 acquires the feature amount data from the gait measurement device 10. The data acquisition unit 131 outputs the received feature amount data to the estimation unit 133. The data acquisition unit 131 may receive the feature amount data from the gait measurement device 10 via a wire such as a cable, or may receive the feature amount data from the gait measurement device 10 via wireless communication. For example, the data acquisition unit 131 is configured to receive the feature amount data from the gait measurement device 10 via a wireless communication function (not illustrated) conforming to a standard such as Bluetooth (registered trademark) or Wi-Fi (registered trademark). The communication function of the data acquisition unit 131 may conform to a standard other than Bluetooth (registered trademark) or Wi-Fi (registered trademark).

The storage unit 132 stores an estimation model for estimating the grip strength as the muscular strength index using the feature amount data extracted from the gait waveform data. The storage unit 132 stores an estimation model that has machine-learned the relationship between the feature amount data regarding the grip strengths of a plurality of subjects and the grip strength. For example, the storage unit 132 stores an estimation model for estimating the grip strength machine-learned regarding a plurality of subjects. The gait phase cluster from which the feature amount data used for the estimation of the grip strength is extracted differs depending on the sex. Therefore, the storage unit 132 may store a male estimation model and a female estimation model. In other words, the storage unit 132 may store an estimation model according to the attribute.

The storage unit 132 stores an estimation model that estimates the knee extension strength as the muscular strength index using the estimated grip strength. NPL 1 discloses that there is a correlation between the grip strength and the knee extension strength (NPL 1: R. Bohannon, et al., “Grip and Knee Extension Muscle Strength Reflect a Common Construct among Adults”, Muscle Nerve, 2012 October, vol. 46 (4), pp. 555-558). For example, the storage unit 132 estimates the knee extension strength according to the grip strength using the correlation of the graph disclosed in FIG. 1 of NPL 1. The storage unit 132 may store an estimation model that has machine-learned the relationship between the feature amount data regarding the knee extension strengths of a plurality of subjects and the knee extension strength.

It is sufficient if the estimation model is stored in the storage unit 132 at the time of shipment of a product from a factory, calibration before the user uses the muscular strength index estimation system 1, or the like. For example, an estimation model stored in a storage device such as an external server may be used. In that case, it is sufficient if the estimation model is configured to be used via an interface (not illustrated) connected to the storage device.

The estimation unit 133 acquires the feature amount data from the data acquisition unit 131. The estimation unit 133 executes estimation of the muscular strength index using the acquired feature amount data. The estimation unit 133 inputs the feature amount data to the estimation model stored in the storage unit 132. The estimation unit 133 outputs an estimation result according to the muscular strength index output from the estimation model. In a case where an estimation model stored in an external storage device constructed in a cloud, a server, or the like is used, the estimation unit 133 is configured to use the estimation model via an interface (not illustrated) connected to the storage device.

The output unit 135 outputs an estimation result of the muscular strength index by the estimation unit 133. For example, the output unit 135 displays the estimation result of the muscular strength index on the screen of the mobile terminal of the subject (user). For example, the output unit 135 outputs the estimation result to an external system or the like that uses the estimation result. The use of the muscular strength index output from the muscular strength index estimation device 13 is not particularly limited.

For example, the muscular strength index estimation device 13 is connected to an external system or the like constructed in a cloud or a server via a mobile terminal (not illustrated) carried by a subject (user). The mobile terminal (not illustrated) is a portable communication device. For example, the mobile terminal is a portable communication device having a communication function, such as a smartphone, a smart watch, or a mobile phone. For example, the muscular strength index estimation device 13 is connected to the mobile terminal via a wire such as a cable. For example, the muscular strength index estimation device 13 is connected to the mobile terminal via wireless communication. For example, the muscular strength index estimation device 13 is connected to the mobile terminal via a wireless communication function (not illustrated) conforming to a standard such as Bluetooth (registered trademark) or Wi-Fi (registered trademark). The communication function of the muscular strength index estimation device 13 may conform to a standard other than Bluetooth (registered trademark) or Wi-Fi (registered trademark). The estimation result of the muscular strength index may be used by an application installed in the mobile terminal. In that case, the mobile terminal executes processing using the estimation result by application software or the like installed in the mobile terminal.

### [Estimation of Grip Strength of Male]

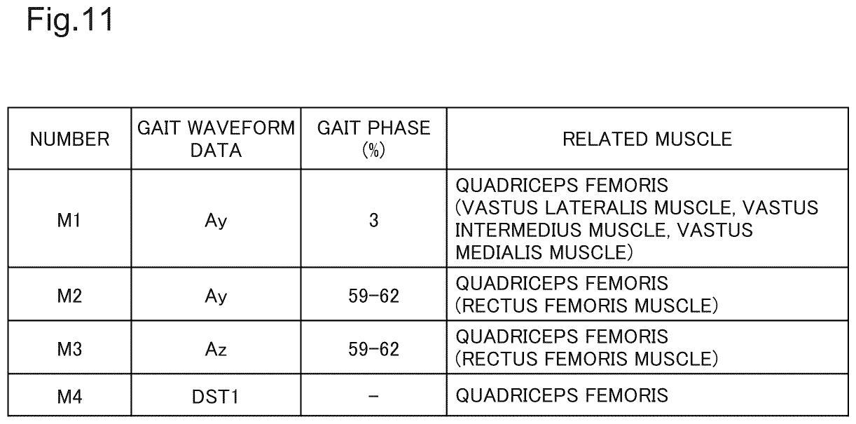

Next, the correlation between the grip strength of a male and the feature amount data will be described with reference to a verification example. FIG. 11 is a correspondence table summarizing feature amounts used to estimate the grip strength of a male. The correspondence table in FIG. 11 associates the number of the feature amount, the gait waveform data from which the feature amount is extracted, the gait phase (%) from which the gait phase cluster is extracted, and the related muscle. In the case of a male, there is a correlation between the activity of quadriceps femoris and the grip strength. Therefore, the feature amounts M1 to M4 extracted from the gait phase in which the feature of the activity of the quadriceps femoris appears are used to estimate the grip strength of the male.

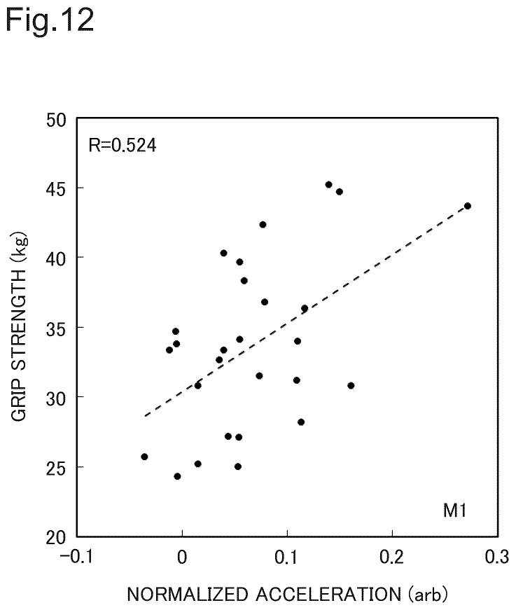

FIGS. 12 to 15 are verification results of the correlation between the grip strength of the male and the feature amount data. With respect to the grip strength of the male, the correlation between the estimation value estimated using the feature amount extracted according to gait with the footwear in which the gait measurement device 10 is mounted and the measurement value (true value) of the grip strength using the grip strength meter was verified for 27 male subjects in the age group of 60 to 85 years old.

The feature amount M1 is extracted from a section of gait phase 3% of gait waveform data Ay related to the time-series data of the traveling-direction acceleration (Y-direction acceleration). The gait phase 3% is included in the load response period T1. The feature amount M1 mainly includes features related to the motion of the vastus lateralis muscle, the vastus intermedius muscle, and the vastus medialis muscle of the quadriceps femoris. FIG. 12 is a verification result of the correlation between the feature amount M1 and the grip strength of the male. The horizontal axis of the graph in FIG. 12 is normalized acceleration. A correlation coefficient R between the feature amount M1 and the grip strength of the male was 0.524.

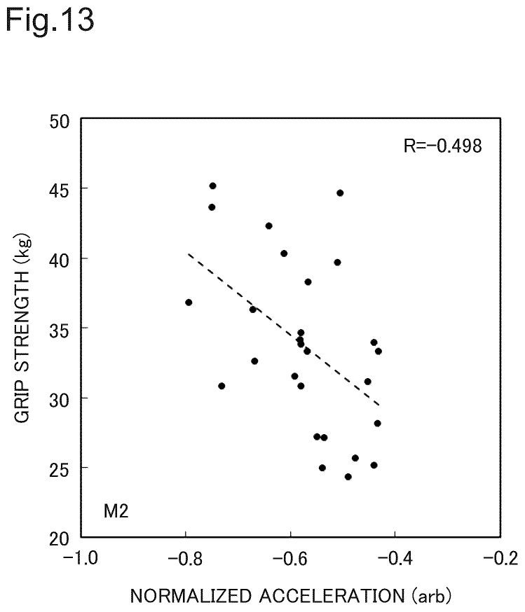

The feature amount M2 is extracted from a section of gait phase 59 to 62% of gait waveform data Ay related to the time-series data of the traveling-direction acceleration (Y-direction acceleration). The gait phase 59 to 62% is included in the pre-swing period T4. The feature amount M2 mainly includes features related to the motion of the rectus femoris muscle of the quadriceps femoris. FIG. 13 is a verification result of the correlation between the feature amount M2 and the grip strength of the male. The horizontal axis of the graph in FIG. 13 is normalized acceleration. A correlation coefficient R between the feature amount M2 and the grip strength of the male was −0.498.

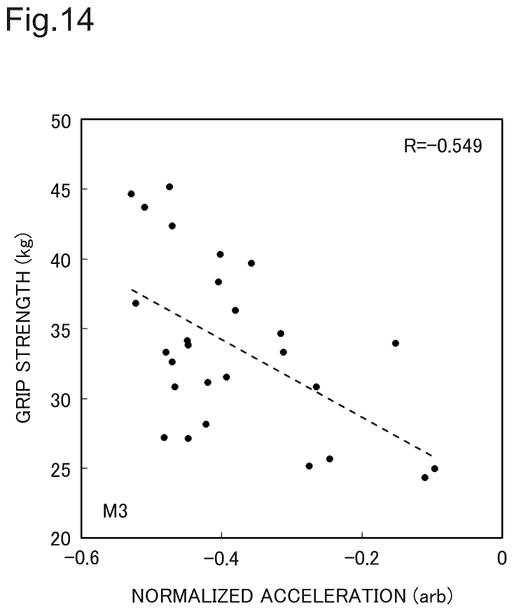

The feature amount M3 is extracted from a section of gait phase 59 to 62% of gait waveform data Az related to the time-series data of the perpendicular-direction acceleration (Z-direction acceleration). The gait phase 59 to 62% is included in the pre-swing period T4. The feature amount M3 mainly includes features related to the motion of the rectus femoris muscle of the quadriceps femoris. FIG. 14 is a verification result of the correlation between the feature amount M3 and the grip strength of the male. The horizontal axis of the graph in FIG. 14 is normalized acceleration. A correlation coefficient R between the feature amount M3 and the grip strength of the male was −0.549.

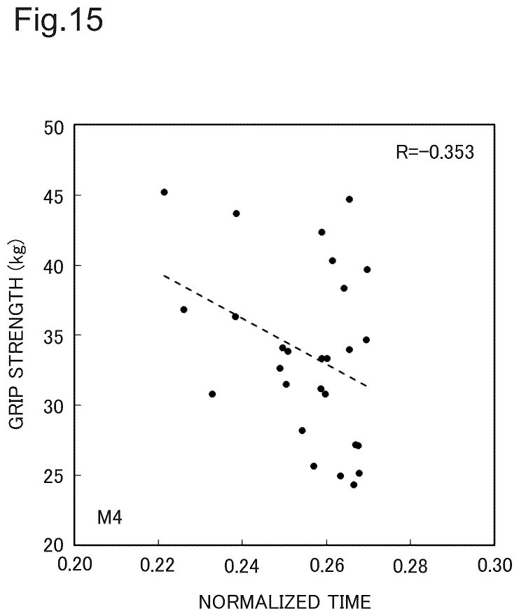

The feature amount M4 is a proportion of a period from the heel contact to the opposite toe off within the period in which both feet simultaneously touch the ground (DST1) (double support time (DST)). DST1 is a proportion of a period from the heel contact to the opposite toe off in one gait cycle. The feature amount M4 mainly includes a feature caused by the quadriceps femoris. FIG. 15 is a verification result of the correlation between the feature amount M4 and the grip strength of the male. The horizontal axis of the graph in FIG. 15 is normalized time. A correlation coefficient R between the feature amount M4 and the grip strength of the male was −0.353.

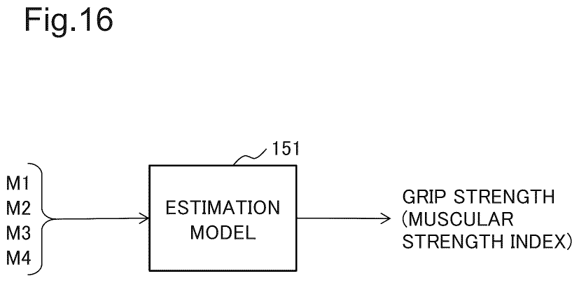

FIG. 16 is a conceptual diagram illustrating an example in which the feature amounts M1 to M4 extracted from the sensor data measured along with gait of the user are input to an estimation model 151 constructed in advance for estimating the grip strength of a male as the muscular strength index, and an estimation value of the grip strength is output. The estimation model 151 (also referred to as the male estimation model) outputs the grip strength as the muscular strength index according to the inputs of the feature amounts M1 to M4. For example, the estimation model 151 is generated by machine learning using training data in which the feature amounts M1 to M4 used for estimating the grip strength of the male are explanatory variables and the grip strength of the male is an objective variable. The estimation result of the estimation model 151 is not limited as long as the estimation result regarding the grip strength as the muscular strength index is output according to the input of the feature amount data for estimating the grip strength of the male. For example, the estimation model 151 may be a model that estimates the grip strength of the male using attributes such as age and height as explanatory variables in addition to the feature amounts M1 to M4 used to estimate the grip strength of the male.

For example, the storage unit 132 stores an estimation model for estimating the grip strength of a male using a multiple regression prediction method. For example, the storage unit 132 stores a parameter for estimating a grip strength GM of the male using Formula 1 described below.

$$GM = a 1 \times M 1 + a 2 \times M 2 + a 3 \times M 3 + a 4 \times M 4 + a 0 \qquad ( 1 )$$

In Formula 1 described above, M1, M2, M3, and M4 are feature amounts for each gait phase cluster used to estimate the grip strength of the male indicated in the correspondence table in FIG. 11. a1, a2, a3, and a4 are coefficients multiplied by M1, M2, M3, and M4, a0 is a constant term. For example, a0, a1, a2, a3, and a4 are stored in the storage unit 132.

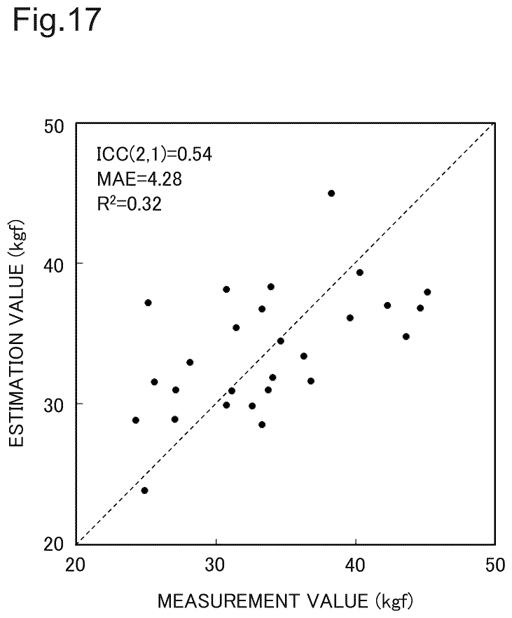

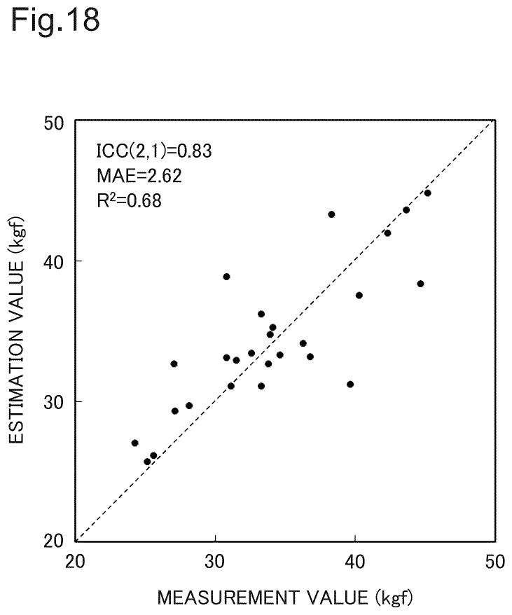

Next, a result of evaluating the estimation model 151 generated using the measurement data of the above-described 27 male subjects will be described. Here, a verification example (FIG. 17) in which the muscular strength index (grip strength) is estimated using the attribute of the male subjects is compared with a verification example (FIG. 18) in which the muscular strength index (grip strength) is estimated using the feature amount of the gait of the male subjects. FIGS. 17 and 18 illustrate the results of testing the estimation model generated using the measurement data of 26 people using the measurement data of the remaining one person by a leave-one-subject-out (LOSO) method. FIGS. 17 and 18 illustrate the results of performing the LOSO on all the (27) subjects and associating the prediction value by the test with the measurement value (true value). The test result of the LOSO was evaluated by values of intraclass correlation coefficients (ICC), a mean absolute error MAE, and a determination coefficient R². As the intraclass correlation coefficient ICC, an intraclass correlation coefficient ICC (2,1) was used in order to evaluate inter-rater reliability.

FIG. 17 illustrates a verification result of an estimation model of a comparative example in which training data in which age, height, and weight are explanatory variables and the grip strength of the male is an objective variable is machine-learned. In the estimation model of the comparative example, the intraclass correlation coefficient ICC (2,1) was 0.54, the mean absolute error MAE was 4.28, and the determination coefficient R² was 0.32.

FIG. 18 illustrates a verification result of the estimation model 151 of the present example embodiment in which training data in which the feature amounts M1 to M4, age, and height are explanatory variables and the grip strength of the male is an objective variable is machine-learned. In the estimation model 151 of the present example embodiment, the intraclass correlation coefficient ICC (2,1) was 0.83, the mean absolute error MAE was 2.62, and the determination coefficient R² was 0.68. That is, the estimation model 151 of the present example embodiment has higher reliability and smaller error than the estimation model of the comparative example, and the objective variable is sufficiently described by the explanatory variable. That is, by the method of the present example embodiment, it is possible to generate the estimation model 151 having high reliability, a small error, and the objective variable sufficiently described by the explanatory variable as compared with the estimation model using only the attribute.

### [Estimation of Grip Strength of Female]

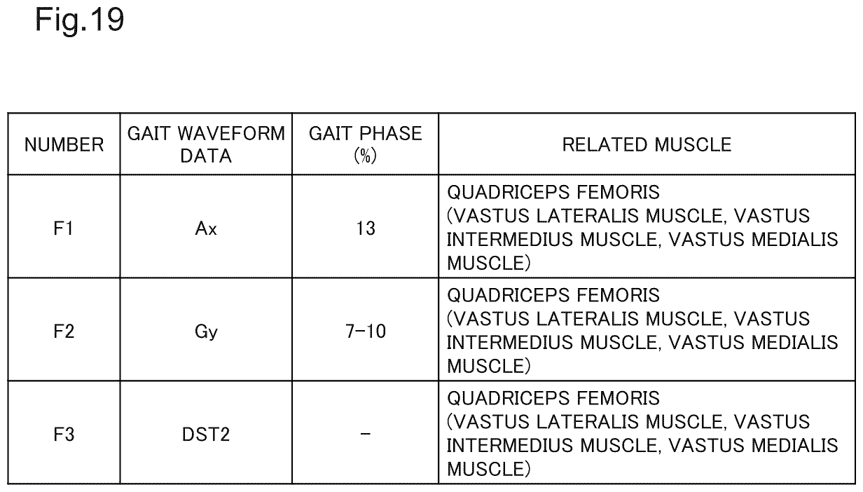

Next, the correlation between the grip strength of a female and the feature amount data will be described with reference to a verification example. FIG. 19 is a correspondence table summarizing feature amounts used to estimate the grip strength of a female. The correspondence table in FIG. 19 associates the number of the feature amount, the gait waveform data from which the feature amount is extracted, the gait phase (%) from which the gait phase cluster is extracted, and the related muscle. In the case of a female, there is a correlation between the activities of the vastus lateralis muscle, the vastus intermedius muscle, and the vastus medialis muscle of the quadriceps femoris and the grip strength. Therefore, the feature amounts F1 to F3 extracted from the gait phase in which the feature of the activities of the vastus lateralis muscle, the vastus intermedius muscle, and the vastus medialis muscle appears are used to estimate the grip strength of the female.

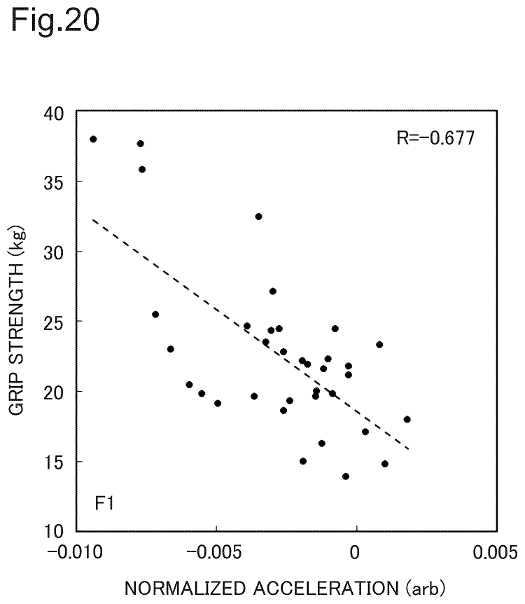

FIGS. 20 to 22 are verification results of the correlation between the grip strength of the female and the feature amount data. With respect to the grip strength of the female, the correlation between the estimation value estimated using the feature amount extracted according to gait with the footwear in which the gait measurement device 10 is mounted and the measurement value (true value) of the grip strength using the grip strength meter was verified for 35 female subjects in the age group of 60 to 85 years old.

The feature amount F1 is extracted from a section of gait phase 13% of gait waveform data Ax related to the time-series data of the lateral-direction acceleration (X-direction acceleration). The gait phase 13% is included in the mid-stance period T2. The feature amount F1 mainly includes features related to the motion of the vastus lateralis muscle, the vastus intermedius muscle, and the vastus medialis muscle of the quadriceps femoris. FIG. 20 is a verification result of the correlation between the feature amount F1 and the grip strength of the female. The horizontal axis of the graph in FIG. 20 is normalized acceleration. A correlation coefficient R between the feature amount F1 and the grip strength of the female was 0.677.

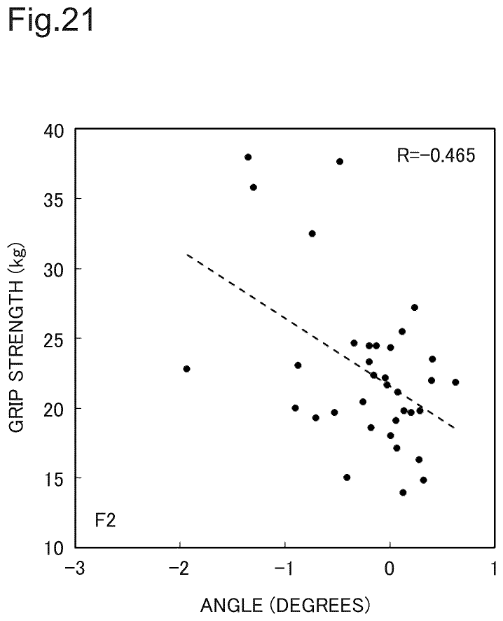

The feature amount F2 is extracted from a section of gait phase 7 to 10% of gait waveform data Gy related to the time-series data of the angular velocity (pitch angular velocity) in the coronal plane (around the Y axis). The gait phase 7 to 10% is included in the load response period T1. The feature amount F2 mainly includes features related to the motion of the vastus lateralis muscle, the vastus intermedius muscle, and the vastus medialis muscle. FIG. 21 is a verification result of the correlation between the feature amount F2 and the grip strength of the female. The horizontal axis of the graph in FIG. 21 is the angle of the sole of the foot in the coronal plane. A correlation coefficient R between the feature amount F2 and the grip strength of the female was-0.465.

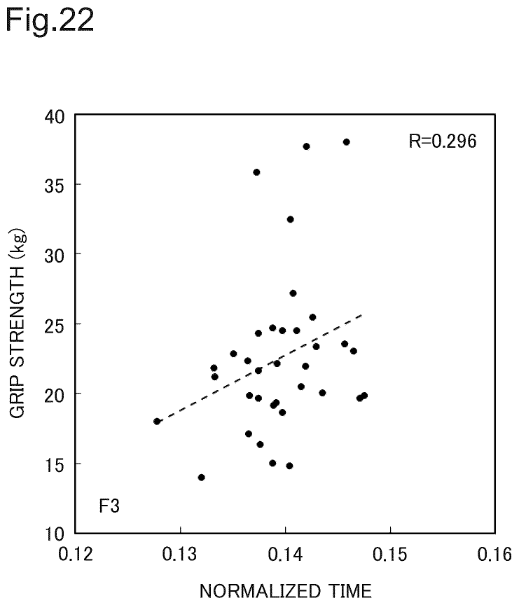

The feature amount F3 is a proportion of a period from the opposite heal strike to the toe off within the period in which both feet simultaneously touch the ground (DST2) (double support time (DST)). DST2 is a proportion of a period from the opposite heal strike to the toe off in one gait cycle. The sum of DST1 and DST2 corresponds to a period in which both feet simultaneously touch the ground in one gait cycle. The feature amount F3 mainly includes features related to the motion of the vastus lateralis muscle, the vastus intermedius muscle, and the vastus medialis muscle. FIG. 22 is a verification result of the correlation between the feature amount F3 and the grip strength of the female. The horizontal axis of the graph in FIG. 22 is normalized time. A correlation coefficient R between the feature amount F3 and the grip strength of the female was 0.296.

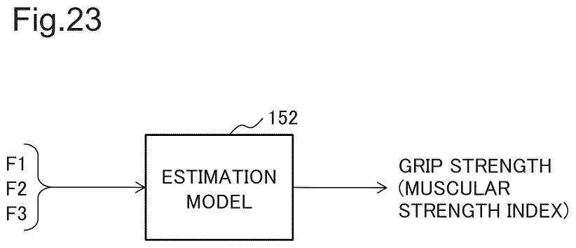

FIG. 23 is a conceptual diagram illustrating an example in which the feature amount data extracted from the sensor data measured along with gait of the user is input to an estimation model 152 constructed in advance for estimating the grip strength of a female as the muscular strength index, and an estimation value of the grip strength is output. The estimation model 152 (also referred to as the female estimation model) outputs the grip strength as the muscular strength index according to the input of the feature amount data. For example, the estimation model 152 is generated by machine learning using training data in which the feature amount data used for estimating the grip strength of the female is an explanatory variable and the grip strength of the female is an objective variable. The estimation result of the estimation model 152 is not limited as long as the estimation result regarding the grip strength as the muscular strength index is output according to the input of the feature amount data for estimating the grip strength of the female. For example, the estimation model 152 may be a model that estimates the grip strength of the female using attributes such as age and height as explanatory variables in addition to the feature amount data used to estimate the grip strength of the female.

For example, the storage unit 132 stores an estimation model for estimating the grip strength of a female using a multiple regression prediction method. For example, the storage unit 132 stores a parameter for estimating a grip strength GF of the female using Formula 2 described below.

$$GF = b 1 \times F 1 + b 2 \times F 2 + b 3 \times F 3 + b 0 \qquad ( 2 )$$

In Formula 1 described above, F1, F2, and F3 are feature amounts for each gait phase cluster used to estimate the grip strength of the female indicated in the correspondence table in FIG. 19. b1, b2, and b3 are coefficients multiplied by F1, F2, and F3. b0 is a constant term. For example, b0, b1, b2, and b3 are stored in the storage unit 132.

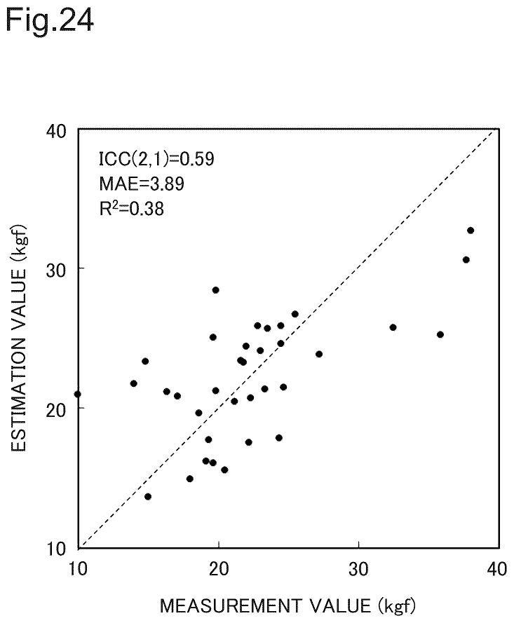

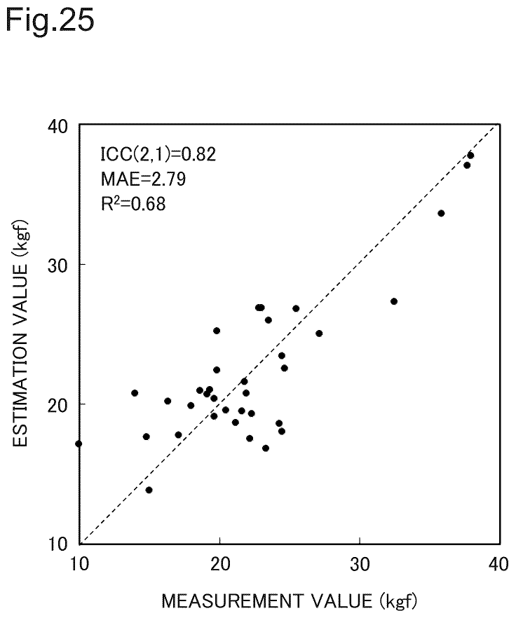

Next, a result of evaluating the estimation model 152 generated using the measurement data of the above-described 35 female subjects will be described. Here, a verification example (FIG. 24) in which the muscular strength index (grip strength) is estimated using the attribute of the female subjects is compared with a verification example (FIG. 25) in which the muscular strength index (grip strength) is estimated using the feature amount of the gait of the female subjects. FIGS. 24 and 25 illustrate the results of testing the estimation model generated using the measurement data of 34 people using the measurement data of the remaining one person by a leave-one-subject-out (LOSO) method. In FIGS. 24 and 25, the LOSO is performed on all the (35) subjects and the prediction value by the test is associated with the measurement value (true value). The test result of the LOSO was evaluated by values of intraclass correlation coefficients (ICC), a mean absolute error MAE, and a determination coefficient R². As the intraclass correlation coefficient ICC, an intraclass correlation coefficient ICC (2,1) was used in order to evaluate inter-rater reliability.

FIG. 24 illustrates a verification result of an estimation model of a comparative example in which training data in which age, height, and weight are explanatory variables and the grip strength of the female is an objective variable is machine-learned. In the estimation model of the comparative example, the intraclass correlation coefficient ICC (2,1) was 0.59, the mean absolute error MAE was 3.89, and the determination coefficient R² was 0.38.

FIG. 25 illustrates a verification result of the estimation model 152 of the present example embodiment in which training data in which the feature amounts F1 to F3, age, and height are explanatory variables and the grip strength of the female is an objective variable is machine-learned. In the estimation model 151 of the present example embodiment, the intraclass correlation coefficient ICC (2,1) was 0.82, the mean absolute error MAE was 2.79, and the determination coefficient R² was 0.68. That is, the estimation model 152 of the present example embodiment has higher reliability and smaller error than the estimation model of the comparative example, and the objective variable is sufficiently described by the explanatory variable. That is, by the method of the present example embodiment, it is possible to generate the estimation model 152 having high reliability, a small error, and the objective variable sufficiently described by the explanatory variable as compared with the estimation model using only the attribute.

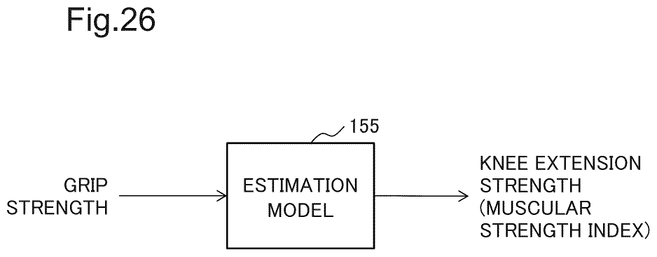

FIG. 26 is an estimation model 155 that outputs the knee extension strength as the muscular strength index according to the input of the grip strength estimated using the estimation model 151 for a male or the estimation model 152 for a female. For example, the estimation model 155 (also referred to as a knee extension strength estimation model) is a model that estimates the knee extension strength according to the grip strength based on the correlation of the graph disclosed in FIG. 1 of NPL 1. By using the estimation model 155, it is possible to estimate the knee extension strength as the muscular strength index using the grip strength estimated using the estimation model 151 for a male or the estimation model 152 for a female. The estimation model 155 may be a model in which training data in which the feature amount data of the estimation model 151 for a male or the estimation model 152 for a female is an explanatory variable and the knee extension strength is an objective variable is machine-learned.

### Operation

Next, an operation of the muscular strength index estimation system 1 will be described with reference to the drawings. Here, the gait measurement device 10 and the muscular strength index estimation device 13 included in the muscular strength index estimation system 1 will be individually described. With respect to the gait measurement device 10, an operation of the feature amount data generation unit 12 included in the gait measurement device 10 will be described.

### [Gait Measurement Device]

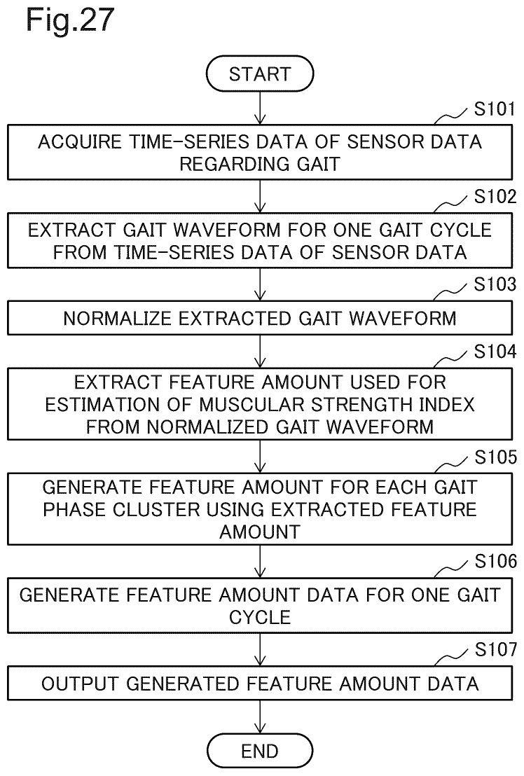

FIG. 27 is a flowchart for describing an operation of the feature amount data generation unit 12 included in the gait measurement device 10. In the description along the flowchart of FIG. 27, the feature amount data generation unit 12 will be described as an operation subject.

In FIG. 27, first, the feature amount data generation unit 12 acquires the time-series data of the sensor data regarding gait (Step S101).

Next, the feature amount data generation unit 12 extracts the gait waveform data for one gait cycle from the time-series data of the sensor data (Step S102). The feature amount data generation unit 12 detects the heel contact and the toe off from the time-series data of the sensor data. The feature amount data generation unit 12 extracts the time-series data of the section between the consecutive pieces of heel contact as gait waveform data for one gait cycle.

Next, the feature amount data generation unit 12 normalizes the extracted gait waveform data for one gait cycle (Step S103). The feature amount data generation unit 12 normalizes the gait waveform data for one gait cycle to a gait cycle of 0 to 100% (first normalization). Further, the feature amount data generation unit 12 normalizes the ratio of the stance phase to the swing phase in the first-normalized gait waveform data for one gait cycle to 60:40 (second normalization).

Next, the feature amount data generation unit 12 extracts a feature amount from the gait phase used for estimation of the muscular strength index with respect to the normalized gait waveform (Step S104). For example, the feature amount data generation unit 12 extracts a feature amount input to an estimation model constructed for each sex. Next, the feature amount data generation unit 12 generates a feature amount for each gait phase cluster using the extracted feature amount (Step S105).

Next, the feature amount data generation unit 12 integrates the feature amounts for each gait phase cluster to generate the feature amount data for one gait cycle (Step S106).

Next, the feature amount data generation unit 12 outputs the generated feature amount data to the muscular strength index estimation device 13 (Step S107).

### [Muscular Strength Index Estimation Device]

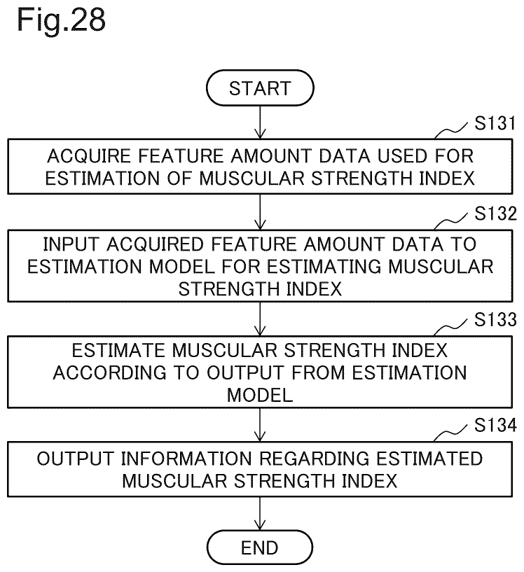

FIG. 28 is a flowchart for describing the operation of the muscular strength index estimation device 13. In the description along the flowchart of FIG. 28, the muscular strength index estimation device 13 will be described as an operation subject.

In FIG. 28, first, the muscular strength index estimation device 13 acquires the feature amount data generated using the sensor data regarding gait (Step S131).

Next, the muscular strength index estimation device 13 inputs the acquired feature amount data to the estimation model for estimating the muscular strength index (Step S132).

Next, the muscular strength index estimation device 13 estimates the muscular strength index of the user according to the output (estimation value) from the estimation model (Step S133). For example, the muscular strength index estimation device 13 estimates the grip strength of the user as the muscular strength index. For example, the muscular strength index estimation device 13 estimates the knee extension strength of the user as the muscular strength index. For example, the muscular strength index estimation device 13 estimates the total whole body muscular strength of the user according to the estimated grip strength.

Next, the muscular strength index estimation device 13 outputs information regarding the estimated muscular strength index (Step S134). For example, the muscular strength index is output to a terminal device (not illustrated) carried by the user. For example, the muscular strength index is output to a system that executes processing using the muscular strength index.

### Application Example

Next, an application example according to the present example embodiment will be described with reference to the drawings. In the application example described below, an example in which the function of the muscular strength index estimation device 13 installed in the mobile terminal carried by the user estimates the muscular strength index using the feature amount data measured by the gait measurement device 10 arranged in a shoe will be described.

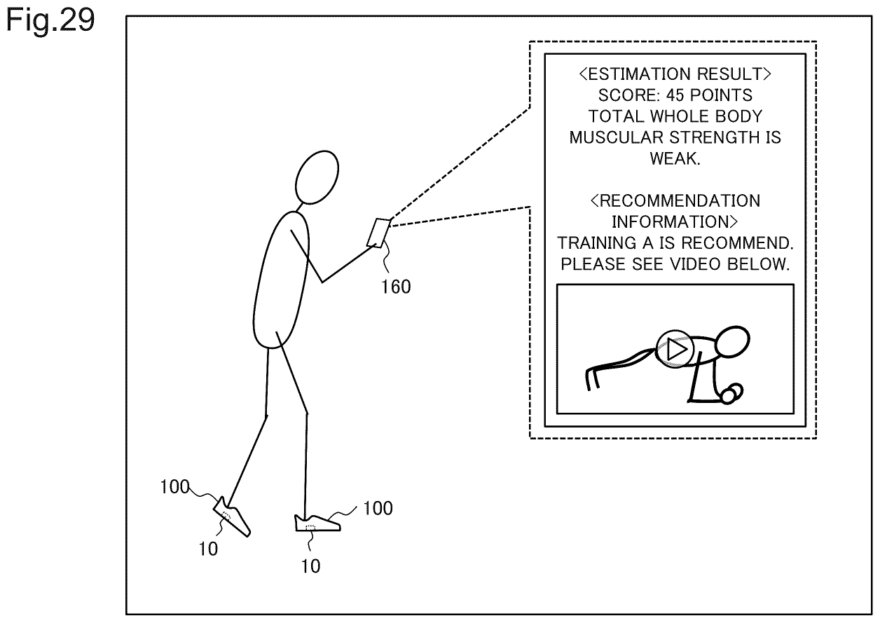

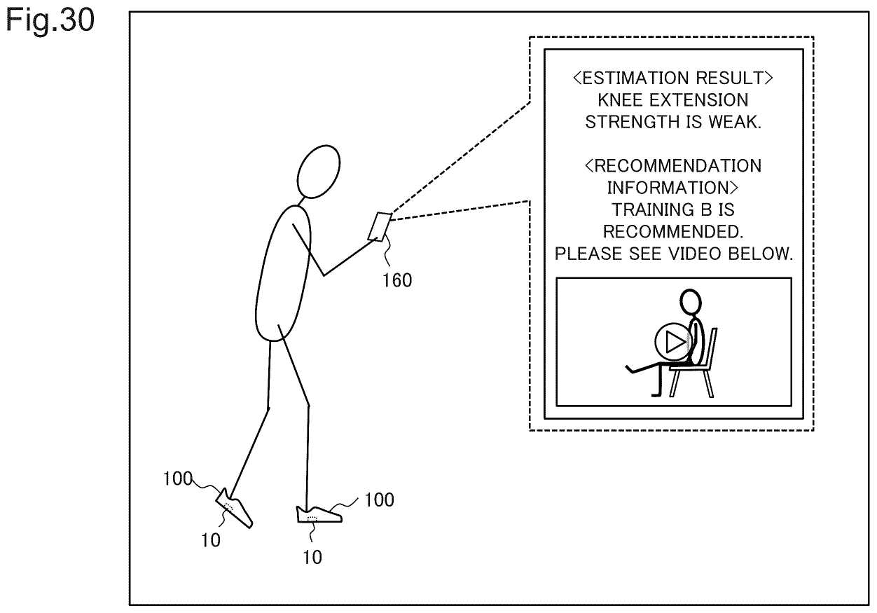

FIGS. 29 to 30 are conceptual diagrams illustrating an example in which the estimation result by the muscular strength index estimation device 13 is displayed on the screen of a mobile terminal 160 carried by the user walking while wearing the shoe 100 in which the gait measurement device 10 is arranged. FIGS. 29 to 30 are examples in which information according to the estimation result of the muscular strength index using the feature amount data according to the sensor data measured during the gait of the user is displayed on the screen of the mobile terminal 160.

FIG. 29 is an example in which information related to the estimation value of the grip strength as the muscular strength index is displayed on the screen of the mobile terminal 160. In the example of FIG. 29, a score quantified based on a preset criterion is displayed on the display unit of the mobile terminal 160 as the estimation result of the total whole body muscular strength. In the example of FIG. 29, information regarding the estimation result of the total whole body muscular strength “total whole body muscular strength is weak” is displayed on the display unit of the mobile terminal 160 according to the estimation value of the grip strength as the muscular strength index. In the example of FIG. 29, according to the estimation value of the grip strength as the muscular strength index, recommendation information according to the estimation result of the total whole body muscular strength “Training A is recommended. Please see the video below” is displayed on the display unit of the mobile terminal 160. The user who has confirmed the information displayed on the display unit of the mobile terminal 160 can practice the training leading to an increase in the total whole body muscular strength by exercising with reference to the video of the training A according to the recommendation information.

FIG. 30 is an example in which information related to the estimation value of the knee extension strength as the muscular strength index is displayed on the screen of the mobile terminal 160. In the example of FIG. 30, information regarding the estimation result of the knee extension strength “knee extension strength is weak” is displayed on the display unit of the mobile terminal 160 according to the estimation value of the knee extension strength as the muscular strength index. For example, a score quantified based on a preset criterion may be displayed on the display unit of the mobile terminal 160 as the estimation result of the knee extension strength. In the example of FIG. 30, according to the estimation value of the knee extension strength as the muscular strength index, recommendation information according to the estimation result of the knee extension strength “Training B is recommended. Please see the video below” is displayed on the display unit of the mobile terminal 160. The user who has confirmed the information displayed on the display unit of the mobile terminal 160 can practice the training leading to an increase in the knee extension strength by exercising with reference to the video of the training B according to the recommendation information.

As described above, the muscular strength index estimation system of the present example embodiment includes the gait measurement device and the muscular strength index estimation device. The gait measurement device includes the sensor and the feature amount data generation unit. The sensor includes the acceleration sensor and the angular velocity sensor. The sensor measures a spatial acceleration using the acceleration sensor. The sensor measures a spatial angular velocity using the angular velocity sensor. The sensor generates sensor data regarding the motion of the foot by using the measured spatial acceleration and spatial angular velocity. The sensor outputs the generated sensor data to the feature amount data generation unit. The feature amount data generation unit acquires the time-series data of the sensor data regarding the motion of the foot. The feature amount data generation unit extracts the gait waveform data for one gait cycle from the time-series data of the sensor data. The feature amount data generation unit normalizes the extracted gait waveform data. The feature amount data generation unit extracts, from the normalized gait waveform data, a feature amount related to the muscular strength index of an estimation target from the gait phase cluster including at least one temporally continuous gait phase. The feature amount data generation unit generates the feature amount data including the extracted feature amount. The feature amount data generation unit outputs the generated feature amount data.

The muscular strength index estimation device includes the data acquisition unit, the storage unit, the estimation unit, and the output unit. The data acquisition unit acquires the feature amount data including the feature amount extracted from the feature of the gait of the user and used for estimating the muscular strength index of the user. The storage unit stores the estimation model that outputs the muscular strength index according to the input of the feature amount data. The estimation unit inputs the acquired feature amount data to the estimation model to estimate the muscular strength index of the user. The output unit outputs information regarding the estimated muscular strength index.

The muscular strength index estimation system of the present example embodiment estimates the muscular strength index of the user using the feature amount extracted from the feature of the gait of the user. Therefore, with the muscular strength index estimation system of the present example embodiment, the muscular strength index can be appropriately estimated in everyday life without using an instrument for measuring the muscular strength.

In one aspect of the present example embodiment, the data acquisition unit acquires the feature amount data including the feature amount extracted from the gait waveform data generated using the time-series data of the sensor data regarding the motion of the foot and used to estimate the grip strength as the muscular strength index. According to the present aspect, the muscular strength index can be appropriately estimated in everyday life without using an instrument for measuring the muscular strength by using the sensor data regarding the motion of the foot.

In one aspect of the present example embodiment, the storage unit stores the estimation model generated by machine learning using training data regarding a plurality of subjects. The estimation model is generated by machine learning using training data in which the feature amount extracted from the gait waveform data and used for estimation of the muscular strength index regarding the grip strength is an explanatory variable and the muscular strength index regarding the grip strength of the subject is an objective variable. The estimation unit inputs the feature amount data acquired regarding the user to the estimation model to estimate the muscular strength index regarding the grip strength of the user. According to the present aspect, the muscular strength index regarding the grip strength can be appropriately estimated in everyday life without using an instrument for measuring the grip strength.

In one aspect of the present example embodiment, the storage unit stores the estimation model that has machine-learned using the explanatory variables including the age and height of the subject. The estimation unit inputs the feature amount data, age, and height regarding the user to the estimation model to estimate the muscular strength index regarding the grip strength of the user. In the present aspect, the muscular strength index is estimated including the age and the height that affect the muscular strength index. Therefore, according to the present aspect, the muscular strength index can be measured with higher accuracy.

In one aspect of the present example embodiment, the storage unit stores the male estimation model generated by machine learning using training data regarding a plurality of male subjects. The male estimation model is a model generated by machine learning using training data in which the feature amount extracted from the load response period and the pre-swing period of the gait waveform data and related to the activity of the quadriceps femoris is an explanatory variable and the muscular strength index regarding the grip strength of the male subject is an objective variable. The estimation unit inputs the feature amount data acquired according to the gait of the male user to the male estimation model to estimate the muscular strength index of the male user. According to the present aspect, the muscular strength index of the male user can be estimated with higher accuracy by using the male estimation model customized for males.

In one aspect of the present example embodiment, the storage unit stores the male estimation model generated by machine learning using training data regarding a plurality of male subjects, in which a plurality of feature amounts extracted from gait waveform data are explanatory variables and the muscular strength index regarding the grip strength of the male subjects is an objective variable. The feature amount extracted from the load response period of the gait waveform data of the traveling-direction acceleration and the feature amount extracted from the pre-swing period of the gait waveform data of the traveling-direction acceleration and the perpendicular-direction acceleration are used as explanatory variables. The feature amount regarding the proportion of the period from the heel contact to the opposite toe off in one gait cycle is used as an explanatory variable. The data acquisition unit acquires the feature amount data including the feature amount extracted according to the gait of the male user. The data acquisition unit acquires the feature amount in the load response period of the gait waveform data of the traveling-direction acceleration, the feature amount in the pre-swing period of the gait waveform data of the traveling-direction acceleration and the perpendicular-direction acceleration, and the feature amount related to the proportion of the period from the heel contact to the opposite toe off in one gait cycle. The estimation unit inputs the acquired feature amount data to the male estimation model to estimate the muscular strength index of the male user. According to the present aspect, the muscular strength index of the male user can be estimated with higher accuracy by using the male estimation model customized for males.

In one aspect of the present example embodiment, the storage unit stores the female estimation model generated by machine learning using training data regarding a plurality of female subjects. The female estimation model is a model generated by machine learning using training data in which the feature amount extracted from the load response period of the gait waveform data and related to the activity of the quadriceps femoris is an explanatory variable and the muscular strength index regarding the grip strength of the female subject is an objective variable. The estimation unit inputs the feature amount data acquired according to the gait of the female user to the female estimation model to estimate the muscular strength index of the female user. According to the present aspect, the muscular strength index of the female user can be estimated with higher accuracy by using the female estimation model customized for females.

In one aspect of the present example embodiment, the storage unit stores the female estimation model generated by machine learning using training data regarding a plurality of female subjects, in which a plurality of feature amounts extracted from gait waveform data are explanatory variables and the muscular strength index regarding the grip strength of the female subject is an objective variable. The feature amount extracted from the load response period of the gait waveform data of the lateral-direction acceleration and the feature amount extracted from the gait waveform data of the angular velocity in the coronal plane are used as explanatory variables. The feature amount regarding the proportion of the period from the opposite heal strike to the toe off in one gait cycle is used as an explanatory variable. The data acquisition unit acquires the feature amount data including the feature amount extracted according to the gait of the female user. The data acquisition unit acquires the feature amount data including the feature amount in the load response period of the gait waveform data of the lateral-direction acceleration, the feature amount of the gait waveform data of the angular velocity in the coronal plane, and the feature amount related to the proportion of the period from the opposite heal strike to the toe off in one gait cycle. The estimation unit inputs the acquired feature amount data to the female estimation model to estimate the muscular strength index of the female user. According to the present aspect, the muscular strength index of the female user can be estimated with higher accuracy by using the female estimation model customized for females.

In one aspect of the present example embodiment, the estimation unit estimates the score of the total whole body muscular strength according to the grip strength estimated for the user. The output unit outputs the score of the estimated total whole body muscular strength. According to the present aspect, it is possible to estimate the score of the total whole body muscular strength according to the muscular strength index estimated according to the feature of gait without using an instrument for measuring the muscular strength.

In one aspect of the present example embodiment, the storage unit stores the knee extension strength estimation model that outputs the knee extension strength according to the input of the grip strength, and the estimation unit inputs the grip strength estimated for the user to the knee extension strength estimation model to estimate the knee extension strength of the user as the muscular strength index. According to the present aspect, it is possible to estimate the knee extension strength according to the grip strength estimated according to the feature of the gait without using an instrument for measuring the muscular strength.

In one aspect of the present example embodiment, the storage unit stores the knee extension strength estimation model generated by machine learning using training data regarding a plurality of subjects. The knee extension strength estimation model is a model generated by machine learning using training data in which the feature amount extracted from the gait waveform data and used for estimation of the muscular strength index regarding the knee extension strength is an explanatory variable and the muscular strength index regarding the knee extension strength of the subject is an objective variable. The estimation unit inputs the acquired feature amount data to the knee extension strength estimation model to estimate the muscular strength index regarding the knee extension strength of the user. According to the present aspect, it is possible to estimate the knee extension strength according to the feature of the gait without using an instrument for measuring the muscular strength.

In one aspect of the present example embodiment, the muscular strength index estimation device is mounted on a terminal device having a screen visually recognizable by the user. For example, the muscular strength index estimation device displays information regarding the muscular strength index estimated according to the motion of the foot of the user on the screen of the terminal device. For example, the muscular strength index estimation device displays recommendation information according to the muscular strength index estimated according to the motion of the foot of the user on the screen of the terminal device. For example, the muscular strength index estimation device displays, on the screen of the terminal device, a video related to training for building up a body part related to the muscular strength index as recommendation information according to the muscular strength index estimated according to the motion of the foot of the user. According to the present aspect, the muscular strength index estimated according to the feature of the gait of the user is displayed on the screen visually recognizable by the user in such a way that the user can confirm the information according to the muscular strength state of the user.

### Second Example Embodiment

Next, a machine learning system according to the second example embodiment will be described with reference to the drawings. The machine learning system of the present example embodiment generates an estimation model for estimating the muscular strength index according to the input of the feature amount by machine learning using the feature amount data extracted from the sensor data measured by the gait measurement device.

### Configuration

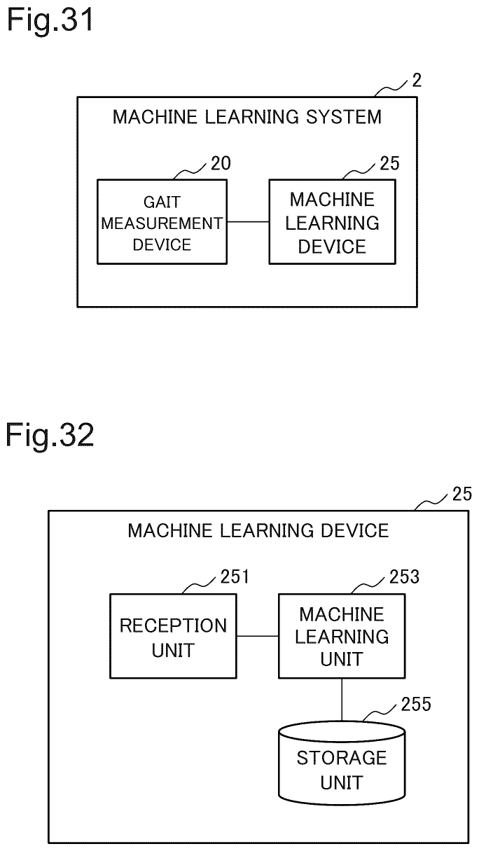

FIG. 31 is a block diagram illustrating an example of a configuration of a machine learning system 2 according to the present example embodiment. The machine learning system 2 includes a gait measurement device 20 and a machine learning device 25. The gait measurement device 20 and the machine learning device 25 may be connected by wire or wirelessly. The gait measurement device 20 and the machine learning device 25 may be configured by a single device. The machine learning system 2 may include only the machine learning device 25 by excluding the gait measurement device 20 from the configuration of the machine learning system 2. Although only one gait measurement device 20 is illustrated in FIG. 31, one gait measurement device 20 may be arranged on each of the left and right feet (two in total). The machine learning device 25 may be configured not to be connected to the gait measurement device 20 but to execute machine learning using the feature amount data generated in advance by the gait measurement device 20 and stored in a database.

The gait measurement device 20 is installed on at least one of the left and right feet. The gait measurement device 20 has the same configuration as the gait measurement device 10 of the first example embodiment. The gait measurement device 20 includes an acceleration sensor and an angular velocity sensor. The gait measurement device 20 converts the measured physical quantity into digital data (also referred to as sensor data). The gait measurement device 20 generates normalized gait waveform data for one gait cycle from time-series data of the sensor data. The gait measurement device 20 generates feature amount data used for estimating the muscular strength index. The gait measurement device 20 transmits the generated feature amount data to the machine learning device 25. The gait measurement device 20 may be configured to transmit the feature amount data to a database (not illustrated) accessed by the machine learning device 25. The feature amount data accumulated in the database is used for machine learning of the machine learning device 25.

The machine learning device 25 receives the feature amount data from the gait measurement device 20. When using the feature amount data accumulated in the database (not illustrated), the machine learning device 25 receives the feature amount data from the database. The machine learning device 25 executes machine learning using the received feature amount data. For example, the machine learning device 25 machine-learns training data in which the feature amount data extracted the gait waveform data of a plurality of subjects is an explanatory variable and a value related to the muscular strength index according to the feature amount data is an objective variable. The machine learning algorithm executed by the machine learning device 25 is not particularly limited. The machine learning device 25 generates an estimation model that has machine-learned using training data regarding a plurality of subjects. The machine learning device 25 stores the generated estimation model. The estimation model that has machine-learned by the machine learning device 25 may be stored in a storage device outside the machine learning device 25.

### [Machine Learning Device]

Next, details of the machine learning device 25 will be described with reference to the drawings. FIG. 32 is a block diagram illustrating an example of a detailed configuration of the machine learning device 25. The machine learning device 25 includes a reception unit 251, a machine learning unit 253, and a storage unit 255.

The reception unit 251 receives the feature amount data from the gait measurement device 20. The reception unit 251 outputs the received feature amount data to the machine learning unit 253. The reception unit 251 may receive the feature amount data from the gait measurement device 20 via a wire such as a cable, or may receive the feature amount data from the gait measurement device 20 via wireless communication. For example, the reception unit 251 is configured to receive the feature amount data from the gait measurement device 20 via a wireless communication function (not illustrated) conforming to a standard such as Bluetooth (registered trademark) or Wi-Fi (registered trademark). The communication function of the reception unit 251 may conform to a standard other than Bluetooth (registered trademark) or Wi-Fi (registered trademark).

The machine learning unit 253 acquires the feature amount data from the reception unit 251. The machine learning unit 253 executes machine learning using the acquired feature amount data. For example, the machine learning unit 253 machine-learns a data set in which the feature amount data extracted with respect to the gait of the subject is an explanatory variable and the grip strength of the subject is an objective variable as training data. For example, the machine learning unit 253 generates an estimation model that estimates the grip strength according to the input of the feature amount data machine-learned regarding a plurality of users. For example, the machine learning unit 253 generates an estimation model that estimates the knee extension strength according to the input of the grip strength machine-learned regarding a plurality of subjects. For example, the machine learning unit 253 generates an estimation model that estimates the knee extension strength according to the input of the feature amount data machine-learned regarding a plurality of subjects. For example, the machine learning unit 253 generates an estimation model for a male and a female rice delivery estimation model. For example, the machine learning unit 253 generates an estimation model for estimating the muscular strength index such as the grip strength or the knee extension strength using the feature amount data extracted regarding the gait of the subject and the attribute data including the age and the height of the subject as explanatory variables. The machine learning unit 253 stores the estimation model that has machine-learned regarding a plurality of subjects in the storage unit 255.

For example, the machine learning unit 253 executes machine learning using a linear regression algorithm. For example, the machine learning unit 253 executes machine learning using an algorithm of a support vector machine (SVM). For example, the machine learning unit 253 executes machine learning using an algorithm of Gaussian process regression (GPR). For example, the machine learning unit 253 executes machine learning using an algorithm of random forest (RF). For example, the machine learning unit 253 may execute unsupervised machine learning of classifying a subject who is a generation source of the feature amount data according to the feature amount data. The machine learning algorithm executed by the machine learning unit 253 is not particularly limited.

The machine learning unit 253 may execute machine learning using the gait waveform data for one gait cycle as an explanatory variable. For example, the machine learning unit 253 executes supervised machine learning in which the gait waveform data of the accelerations in the three axial directions, the angular velocities around the three axes, and the angles (attitude angles) around the three axes is an explanatory variable and a correct value of the muscular strength index of an estimation target is an objective variable. For example, in a case where the gait phase is set in increments of 1% in a gait cycle of 0 to 100%, the machine learning unit 253 machine-learns by using 909 explanatory variables.

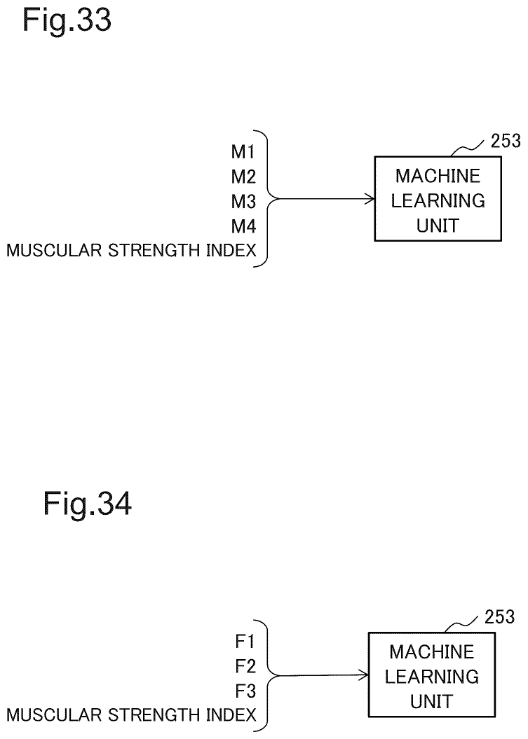

FIG. 33 is a conceptual diagram for describing machine learning for generating an estimation model for a male. FIG. 33 is a conceptual diagram illustrating an example of causing the machine learning unit 253 to machine-learn by using a data set of the feature amounts M1 to M4, which are explanatory variables, and the muscular strength index, which is an objective variable, as training data. For example, the machine learning unit 253 machine-learns data related to a plurality of male subjects, and generates an estimation model that outputs an output (estimation value) related to the muscular strength index of a male according to an input of the feature amount extracted from the sensor data.

FIG. 34 is a conceptual diagram for describing machine learning for generating an estimation model for a female. FIG. 34 is a conceptual diagram illustrating an example of causing the machine learning unit 253 to machine-learn by using a data set of the feature amounts F1 to F3, which are explanatory variables, and the muscular strength index, which is an objective variable, as training data. For example, the machine learning unit 253 machine-learns data related to a plurality of female subjects, and generates an estimation model that outputs an output (estimation value) related to the muscular strength index of a female according to an input of the feature amount extracted from the sensor data.

The storage unit 255 stores the estimation model that has machine-learned regarding a plurality of subjects. For example, the storage unit 255 stores an estimation model for estimating the muscular strength index machine-learned regarding a plurality of subjects. For example, the estimation model stored in the storage unit 255 is used for estimating the muscular strength index by the muscular strength index estimation device 13 of the first example embodiment.

As described above, the machine learning system of the present example embodiment includes the gait measurement device and the machine learning device. The gait measurement device acquires the time-series data of the sensor data regarding the motion of the foot. The gait measurement device extracts gait waveform data for one gait cycle from the time-series data of the sensor data and normalizes the extracted gait waveform data. The gait measurement device extracts, from the normalized gait waveform data, a feature amount related to the muscular strength index of an estimation target from the gait phase cluster including at least one temporally continuous gait phase. The gait measurement device generates the feature amount data including the extracted feature amount. The gait measurement device outputs the generated feature amount data to the machine learning device.

The machine learning device includes the reception unit, the machine learning unit, and the storage unit. The reception unit acquires the feature amount data generated by the gait measurement device. The machine learning unit executes machine learning using the feature amount data. The machine learning unit generates the estimation model that outputs the muscular strength index according to the input of the feature amount (second feature amount) of the gait phase cluster extracted from the time-series data of the sensor data measured along with the gait of the user. The estimation model generated by the machine learning unit is stored in the storage unit.

The machine learning system of the present example embodiment generates an estimation model by using the feature amount data measured by the gait measurement device. Therefore, according to the present aspect, it is possible to generate an estimation model capable of appropriately estimating the muscular strength index in everyday life without using an instrument for measuring the muscular strength.

### Third Example Embodiment

Next, a muscular strength index estimation device according to the third example embodiment will be described with reference to the drawings. The muscular strength index estimation device of the present example embodiment has a simplified configuration of the muscular strength index estimation device included in the muscular strength index estimation system of the first example embodiment.

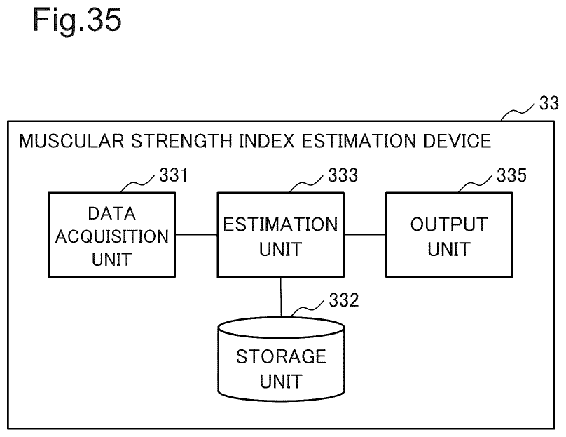

FIG. 35 is a block diagram illustrating an example of a configuration of a muscular strength index estimation device 33 according to the present example embodiment. The muscular strength index estimation device 33 includes a data acquisition unit 331, a storage unit 332, an estimation unit 333, and an output unit 335.

The data acquisition unit 331 acquires the feature amount data including the feature amount extracted from the feature of the gait of the user and used for estimating the muscular strength index of the user. The storage unit 332 stores the estimation model that outputs the muscular strength index according to the input of the feature amount data. The estimation unit 333 inputs the acquired feature amount data to the estimation model to estimate the muscular strength index of the user. The output unit 335 outputs information regarding the estimated muscular strength index.

As described above, according to the present example embodiment, the muscular strength index of the user is estimated using the feature amount extracted from the feature of the gait of the user. Therefore, according to the present example embodiment, the muscular strength index can be appropriately estimated in everyday life without using an instrument for measuring the muscular strength.

### Hardware

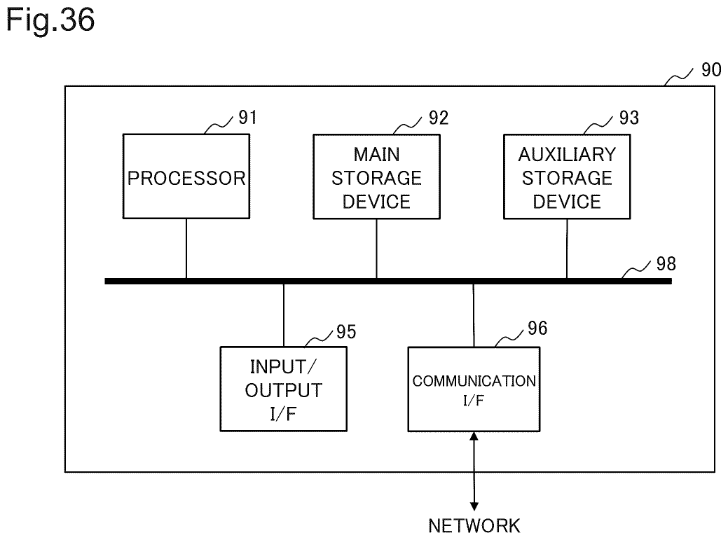

Here, a hardware configuration for executing control and processing according to each example embodiment of the present disclosure will be described using an information processing device 90 of FIG. 36 as an example. The information processing device 90 in FIG. 36 is a configuration example for executing control and processing of each example embodiment, and does not limit the scope of the present disclosure.

As illustrated in FIG. 36, the information processing device 90 includes a processor 91, a main storage device 92, an auxiliary storage device 93, an input/output interface 95, and a communication interface 96. In FIG. 36, the interface is abbreviated as I/F. The processor 91, the main storage device 92, the auxiliary storage device 93, the input/output interface 95, and the communication interface 96 are connected to be capable of data communication via a bus 98. The processor 91, the main storage device 92, the auxiliary storage device 93, and the input/output interface 95 are connected to a network such as the Internet or an intranet via the communication interface 96.

The processor 91 loads a program stored in the auxiliary storage device 93 or the like to the main storage device 92. The processor 91 executes the program loaded on the main storage device 92. In the present example embodiment, it is sufficient if a software program installed in the information processing device 90 is used. The processor 91 executes control and processing according to each example embodiment.

The main storage device 92 has an area in which a program is loaded. A program stored in the auxiliary storage device 93 or the like is loaded to the main storage device 92 by the processor 91. The main storage device 92 is achieved by, for example, a volatile memory such as dynamic random access memory (DRAM). A nonvolatile memory such as magnetoresistive random access memory (MRAM) may be configured/added as the main storage device 92.

The auxiliary storage device 93 stores various data such as a program. The auxiliary storage device 93 is achieved by a local disk such as a hard disk or flash memory. Various data may be stored in the main storage device 92, and the auxiliary storage device 93 may be omitted.

The input/output interface 95 is an interface for connecting the information processing device 90 and a peripheral device based on standards or specifications. The communication interface 96 is an interface for connecting to an external system or device through a network such as the Internet or an intranet based on standards or specifications. The input/output interface 95 and the communication interface 96 may be shared as an interface connected to an external device.

Input devices such as a keyboard, a mouse, and a touch panel may be connected to the information processing device 90 as necessary. These input devices are used to input information and settings. When the touch panel is used as the input device, the display screen of the display device may be configured to serve as the interface of the input device. It is sufficient if data communication between the processor 91 and the input device is mediated by the input/output interface 95.

The information processing device 90 may be provided with a display device for displaying information. In a case where a display device is provided, the information processing device 90 preferably includes a display control device (not illustrated) for controlling display of the display device. It is sufficient if the display device is connected to the information processing device 90 via the input/output interface 95.

The information processing device 90 may be provided with a drive device. The drive device mediates reading of data and a program from a recording medium, writing of a processing result of the information processing device 90 to the recording medium, and the like between the processor 91 and the recording medium (program recording medium). It is sufficient if the drive device is connected to the information processing device 90 via the input/output interface 95.

The above is an example of a hardware configuration for enabling control and processing according to each example embodiment of the present invention. The hardware configuration in FIG. 36 is an example of the hardware configuration for executing control and processing of each example embodiment, and does not limit the scope of the present invention. A program for causing a computer to execute control and processing according to each example embodiment is also included in the scope of the present invention. Further, a program recording medium in which the program according to each example embodiment is recorded is also included in the scope of the present invention. The recording medium can be achieved by, for example, an optical recording medium such as a compact disc (CD) or a digital versatile disc (DVD). The recording medium may be achieved by a semiconductor recording medium such as a universal serial bus (USB) memory or a secure digital (SD) card. The recording medium may be achieved by a magnetic recording medium such as a flexible disk, or another recording medium. When a program executed by the processor is recorded in a recording medium, the recording medium corresponds to a program recording medium.

The components of each example embodiment may be combined in any manner. The components of each example embodiment may be achieved by software or may be achieved by a circuit.

While the invention has been particularly shown and described with reference to example embodiments thereof, the invention is not limited to these embodiments. It will be understood by those of ordinary skill in the art that various changes in form and details may be made therein without departing from the spirit and scope of the present invention as defined by the claims.

Some or all of the above example embodiments may also be described as the following supplementary notes, but are not limited to the following.

### Supplementary Note 1

A muscular strength index estimation device including:
a data acquisition unit that acquires feature amount data including a feature amount, the feature amount being extracted from a feature of gait of a user and being used for estimation of a muscular strength index of the user,
a storage unit that stores an estimation model that outputs the muscular strength index in accordance with an input of the feature amount data,
an estimation unit that inputs the acquired feature amount data to the estimation model to estimate the muscular strength index of the user, and
an output unit that outputs information regarding the estimated muscular strength index.

### Supplementary Note 2

The muscular strength index estimation device according to supplementary note 1, in which
the data acquisition unit
acquires the feature amount data including a feature amount, the feature amount being extracted from gait waveform data generated using time-series data of sensor data regarding a motion of a foot and being used to estimate a grip strength as the muscular strength index.

### Supplementary Note 3

The muscular strength index estimation device according to supplementary note 2, in which
the storage unit
stores the estimation model generated by machine learning using training data regarding a plurality of subjects, in which a feature amount used for estimation of the muscular strength index related to the grip strength extracted from the gait waveform data is an explanatory variable and the muscular strength index related to the grip strength of the subjects is an objective variable, and
the estimation unit
inputs the feature amount data acquired regarding the user to the estimation model to estimate the muscular strength index regarding a grip strength of the user.

### Supplementary Note 4

The muscular strength index estimation device according to supplementary note 3, in which
the storage unit
stores the estimation model that has machine-learned using explanatory variables including age and height of the subject, and
the estimation unit
inputs the feature amount data, age, and height regarding the user to the estimation model to estimate the muscular strength index regarding the grip strength of the user.

### Supplementary Note 5

The muscular strength index estimation device according to supplementary note 3 or 4, in which
the storage unit
stores a male estimation model generated by machine learning using training data regarding a plurality of male subjects, in which a feature amount regarding an activity of a quadriceps femoris extracted from a load response period and a pre-swing period of the gait waveform data is an explanatory variable, and the muscular strength index regarding a grip strength of the male subjects is an objective variable, and
the estimation unit
inputs the feature amount data acquired according to gait of a male user to the male estimation model to estimate the muscular strength index of the male user.

### Supplementary Note 6

The muscular strength index estimation device according to supplementary note 5, in which
the storage unit
stores the male estimation model generated by machine learning using training data regarding the plurality of male subjects, in which a feature amount extracted from a load response period of the gait waveform data of a traveling-direction acceleration, a feature amount extracted from a pre-swing period of the gait waveform data of the traveling-direction acceleration and a perpendicular-direction acceleration, and a feature amount related to a proportion of a period from heel contact to opposite toe off in one gait cycle are explanatory variables, and the muscular strength index related to the grip strength of the male subject is an objective variable,
the data acquisition unit
acquires the feature amount data including the feature amount in the load response period of the gait waveform data of the traveling-direction acceleration, the feature amount in the pre-swing period of the gait waveform data of the traveling-direction acceleration and the perpendicular-direction acceleration, and the feature amount regarding the proportion of the period from heel contact to opposite toe off in one gait cycle, the feature amount data being extracted according to gait of the male user, and
the estimation unit
inputs the acquired feature amount data to the male estimation model to estimate the muscular strength index of the male user.

### Supplementary Note 7

The muscular strength index estimation device according to supplementary note 3 or 4, in which
the storage unit
stores a female estimation model generated by machine learning using training data regarding a plurality of female subjects, in which a feature amount regarding an activity of a quadriceps femoris extracted from a load response period of the gait waveform data is an explanatory variable, and the muscular strength index regarding a grip strength of the female subjects is an objective variable, and
the estimation unit
inputs the feature amount data acquired according to gait of a female user to the female estimation model to estimate the muscular strength index of the female user.

### Supplementary Note 8

The muscular strength index estimation device according to supplementary note 7, in which
the storage unit
stores the female estimation model generated by machine learning using training data regarding the plurality of female subjects, in which a feature amount extracted from a load response period of the gait waveform data of a lateral-direction acceleration, a feature amount extracted from the gait waveform data of an angular velocity in a coronal plane, and a feature amount related to a proportion of a period from opposite heal strike to toe off in one gait cycle are explanatory variables, and the muscular strength index related to the grip strength of the female subject is an objective variable,
the data acquisition unit
acquires the feature amount data including the feature amount in the load response period of the gait waveform data of the lateral-direction acceleration, the feature amount of the gait waveform data of the angular velocity in the coronal plane, and the feature amount regarding the proportion of the period from opposite heal strike to toe off in one gait cycle, the feature amount data being extracted according to gait of the female user, and
the estimation unit
inputs the acquired feature amount data to the female estimation model to estimate the muscular strength index of the female user.

### Supplementary Note 9

The muscular strength index estimation device according to any one of supplementary notes 3 to 8, in which
the estimation unit
estimates a score of total whole body muscular strength according to the grip strength estimated for the user, and
the output unit
outputs the estimated score of the total whole body muscular strength.

### Supplementary Note 10

The muscular strength index estimation device according to any one of supplementary notes 3 to 9, in which
the storage unit
stores a knee extension strength estimation model that outputs a knee extension strength according to an input of the grip strength, and
the estimation unit
inputs the grip strength estimated for the user to the knee extension strength estimation model to estimate the knee extension strength of the user as the muscular strength index.

### Supplementary Note 11

The muscular strength index estimation device according to any one of supplementary notes 3 to 9, in which
the storage unit
stores a knee extension strength estimation model generated by machine learning using training data regarding the plurality of subjects, in which a feature amount used for estimation of the muscular strength index related to a knee extension strength extracted from the gait waveform data is an explanatory variable and the muscular strength index related to the knee extension strength of the subjects is an objective variable, and
the estimation unit
inputs the acquired feature amount data to the knee extension strength estimation model to estimate the muscular strength index regarding the knee extension strength of the user.

### Supplementary Note 12

A muscular strength index estimation system including:
the muscular strength index estimation device according to any one of supplementary notes 1 to 11, and
a gait measurement device including: a sensor that is installed in footwear of a user who is an estimation target of a muscular strength index, measures a spatial acceleration and a spatial angular velocity, generates sensor data regarding a motion of a foot by using the measured spatial acceleration and the measured spatial angular velocity, and outputs the generated sensor data; and a feature amount data generation unit that acquires time-series data of the sensor data including a feature of gait, extracts gait waveform data for one gait cycle from the time-series data of the sensor data, normalizes the extracted gait waveform data, extracts a feature amount used for estimation of the muscular strength index from a gait phase cluster including at least one temporally continuous gait phase from the normalized gait waveform data, generates feature amount data including the extracted feature amount, and outputs the generated feature amount data to the muscular strength index estimation device.

### Supplementary Note 13

The muscular strength index estimation system according to supplementary note 12, in which
the muscular strength index estimation device
is mounted on a terminal device having a screen visually recognizable by the user, and
displays information regarding the muscular strength index estimated according to the motion of the foot of the user on the screen of the terminal device.

### Supplementary Note 14

The muscular strength index estimation system according to supplementary note 13, in which
the muscular strength index estimation device
displays recommendation information according to the muscular strength index estimated according to the motion of the foot of the user on the screen of the terminal device.

### Supplementary Note 15

The muscular strength index estimation system according to supplementary note 14, in which
the muscular strength index estimation device
displays, on the screen of the terminal device, a video related to training for building up a body part related to the muscular strength index as the recommendation information according to the muscular strength index estimated according to the motion of the foot of the user.

### Supplementary Note 16

A muscular strength index estimation method including:
causing a computer to acquire feature amount data including a feature amount, the feature amount being extracted from a feature of gait of a user and being used for estimation of a muscular strength index of the user,
causing the computer to input the acquired feature amount data to an estimation model that outputs the muscular strength index in accordance with an input of the feature amount data to estimate the muscular strength index of the user, and
causing the computer to output information regarding the estimated muscular strength index.

### Supplementary Note 17

A program causing a computer to execute:
processing of acquiring feature amount data including a feature amount from a feature of gait of a user and used for estimation of a muscular strength index of the user,
processing of inputting the acquired feature amount data to an estimation model that outputs the muscular strength index in accordance with an input of the feature amount data to estimate the muscular strength index of the user, and
processing of outputting information regarding the estimated muscular strength index.

### REFERENCE SIGNS LIST

1 muscular strength index estimation system
2 machine learning system
10, 20 gait measurement device
11 sensor
12 feature amount data generation unit
13 muscular strength index estimation device
25 machine learning device
111 acceleration sensor
112 angular velocity sensor
121 acquisition unit
122 normalization unit
123 extraction unit
125 generation unit
127 feature amount data output unit
131, 331 data acquisition unit
132, 332 storage unit
133, 333 estimation unit
135, 335 output unit
251 reception unit
253 machine learning unit
255 storage unit

What is claimed is:

1. A muscular strength index estimation device comprising:
a storage configured to store an estimation model that outputs a muscular strength index in accordance with an input of feature amount data used for estimation of a muscular strength index;
a memory storing instructions; and
a processor connected to the memory and configured to execute the instructions to:
acquire feature amount data including a feature amount extracted from a feature of gait of a user and used for estimation of the muscular strength index of the user;
input the acquired feature amount data to the estimation model to estimate the muscular strength index of the user; and
output information regarding the estimated muscular strength index.

2. The muscular strength index estimation device according to claim 1, wherein
the processor is configured to execute the instructions to
acquire the feature amount data including a feature amount, the feature amount being extracted from gait waveform data generated using time-series data of sensor data regarding a motion of a foot and being used to estimate a grip strength as the muscular strength index.

3. The muscular strength index estimation device according to claim 2, wherein
the storage
stores the estimation model generated by machine learning using training data regarding a plurality of subjects, in which a feature amount used for estimation of the muscular strength index related to the grip strength extracted from the gait waveform data is an explanatory variable and the muscular strength index related to the grip strength of the subjects is an objective variable, and
the processor is configured to execute the instructions to
input the feature amount data acquired regarding the user to the estimation model and estimate the muscular strength index regarding a grip strength of the user.

4. The muscular strength index estimation device according to claim 3, wherein
the storage
stores the estimation model that has machine-learned using explanatory variables including age and height of the subject, and
the processor is configured to execute the instructions to
input the feature amount data, age, and height regarding the user to the estimation model to estimate the muscular strength index regarding the grip strength of the user.

5. The muscular strength index estimation device according to claim 3, wherein
the storage
stores a male estimation model generated by machine learning using training data regarding a plurality of male subjects, in which a feature amount regarding an activity of a quadriceps femoris extracted from a load response period and a pre-swing period of the gait waveform data is an explanatory variable, and the muscular strength index regarding a grip strength of the male subjects is an objective variable, and
the processor is configured to execute the instructions to
inputs the feature amount data acquired according to gait of a male user to the male estimation model to estimate the muscular strength index of the male user.

6. The muscular strength index estimation device according to claim 5, wherein
the storage
stores the male estimation model generated by machine learning using training data regarding the plurality of male subjects, in which a feature amount extracted from a load response period of the gait waveform data of a traveling-direction acceleration, a feature amount extracted from a pre-swing period of the gait waveform data of the traveling-direction acceleration and a perpendicular-direction acceleration, and a feature amount related to a proportion of a period from heel contact to opposite toe off in one gait cycle are explanatory variables, and the muscular strength index related to the grip strength of the male subjects is an objective variable,
the processor is configured to execute the instructions to
acquire the feature amount data including the feature amount in the load response period of the gait waveform data of the traveling-direction acceleration, the feature amount in the pre-swing period of the gait waveform data of the traveling-direction acceleration and the perpendicular-direction acceleration, and the feature amount regarding the proportion of the period from heel contact to opposite toe off in one gait cycle, the feature amount data being extracted according to gait of the male user, and
input the acquired feature amount data to the male estimation model to estimate the muscular strength index of the male user.

7. The muscular strength index estimation device according to claim 3, wherein
the storage
stores a female estimation model generated by machine learning using training data regarding a plurality of female subjects, in which a feature amount regarding an activity of a quadriceps femoris extracted from a load response period of the gait waveform data is an explanatory variable, and the muscular strength index regarding grip strength of the female subjects is an objective variable, and
the processor is configured to execute the instructions to
input the feature amount data acquired according to gait of a female user to the female estimation model to estimate the muscular strength index of the female user.

8. The muscular strength index estimation device according to claim 7, wherein
the storage
stores the female estimation model generated by machine learning using training data regarding the plurality of female subjects, in which a feature amount extracted from a load response period of the gait waveform data of a lateral-direction acceleration, a feature amount extracted from the gait waveform data of an angular velocity in a coronal plane, and a feature amount related to a proportion of a period from opposite heal strike to toe off in one gait cycle are explanatory variables, and the muscular strength index related to the grip strength of the female subject is an objective variable,
the processor is configured to execute the instructions to
acquire the feature amount data including the feature amount in the load response period of the gait waveform data of the lateral-direction acceleration, the feature amount of the gait waveform data of the angular velocity in the coronal plane, and the feature amount regarding the proportion of the period from opposite heal strike to toe off in one gait cycle, the feature amount data being extracted according to gait of the female user, and
input the acquired feature amount data to the female estimation model to estimate the muscular strength index of the female user.

9. The muscular strength index estimation device according to claim 3, wherein
the estimation means is configured to the processor is configured to execute the instructions to
estimate a score of total whole body muscular strength according to the grip strength estimated for the user, and
output the estimated score of the total whole body muscular strength.

10. The muscular strength index estimation device according to claim 3, wherein
the storage
stores a knee extension strength estimation model that outputs a knee extension strength according to an input of the grip strength, and
the processor is configured to execute the instructions to
input the grip strength estimated for the user to the knee extension strength estimation model to estimate the knee extension strength of the user as the muscular strength index.

11. The muscular strength index estimation device according to claim 3, wherein
the storage
stores a knee extension strength estimation model generated by machine learning using training data regarding the plurality of subjects, in which a feature amount used for estimation of the muscular strength index related to a knee extension strength extracted from the gait waveform data is an explanatory variable and the muscular strength index related to the knee extension strength of the subjects is an objective variable, and
the processor is configured to execute the instructions to
input the acquired feature amount data to the knee extension strength estimation model to estimate the muscular strength index regarding the knee extension strength of the user.

12. A muscular strength index estimation system comprising:
the muscular strength index estimation device according to claim 1; and
a gait measurement device including:
a sensor that is installed in footwear of a user who is an estimation target of a muscular strength index, measures a spatial acceleration and a spatial angular velocity, generates sensor data regarding a motion of a foot by using the measured spatial acceleration and the measured spatial angular velocity, and outputs the generated sensor data; and
a memory storing instructions; and
a processor connected to the memory and configured to execute the instructions to:
acquire time-series data of the sensor data including a feature of gait,
extract gait waveform data for one gait cycle from the time-series data of the sensor data,
normalize the extracted gait waveform data,
extract a feature amount used for estimation of the muscular strength index from a gait phase cluster including at least one temporally continuous gait phase from the normalized gait waveform data,
generate feature amount data including the extracted feature amount, and
output the generated feature amount data to the muscular strength index estimation device.

13. The muscular strength index estimation system according to claim 12, wherein
the muscular strength index estimation device is configured to
be mounted on a terminal device having a screen visually recognizable by the user, and
the processer of the muscular strength index estimation device is configured to execute the instructions to
display information regarding the muscular strength index estimated according to the motion of the foot of the user on the screen of the terminal device.

14. The muscular strength index estimation system according to claim 13, wherein
the processer of the muscular strength index estimation device is configured to execute the instructions to
display recommendation information according to the muscular strength index estimated according to the motion of the foot of the user on the screen of the terminal device.

15. The muscular strength index estimation system according to claim 14, wherein
the processer of the muscular strength index estimation device is configured to execute the instructions to
display, on the screen of the terminal device, a video related to training for building up a body part related to the muscular strength index as the recommendation information according to the muscular strength index estimated according to the motion of the foot of the user.

16. A muscular strength index estimation method comprising:
causing a computer to acquire feature amount data including a feature amount, the feature amount being extracted from a feature of gait of a user and being used for estimation of a muscular strength index of the user,
causing the computer to input the acquired feature amount data to an estimation model that outputs the muscular strength index in accordance with an input of the feature amount data to estimate the muscular strength index of the user, and
causing the computer to output information regarding the estimated muscular strength index.

17. A non-transitory recording medium recording a program for causing a computer to execute:
processing of acquiring feature amount data including a feature amount, the feature amount being extracted from a feature of gait of a user and being used for estimation of a muscular strength index of the user,
processing of inputting the acquired feature amount data to an estimation model that outputs the muscular strength index in accordance with an input of the feature amount data to estimate the muscular strength index of the user, and
processing of outputting information regarding the estimated muscular strength index.

18. The muscular strength index estimation system according to claim 14, wherein
the processor of the muscular strength index estimation device is configured to execute the instructions to
display the recommendation information that supports the user for making decision about taking an action on the screen of the terminal device.
